(19) United States
(12) Patent Application Publication
(10) Pub. No.: US 2009/0005338 A1
(43) Pub. Date: Jan. 1, 2009

(54) Carbonyl-substituted titanocenes

(75) Inventors: Andreas Gansauer, Swisttal (DE); Aram Prokop, Berlin (DE); Anja Selig, Berlin (DE); Dieter Franke, Bad Honnef (DE)

Correspondence Address: STERNE, KESSLER, GOLDSTEIN & FOX P.L.L.C., 1100 NEW YORK AVENUE, N.W., WASHINGTON, DC, 20005, US

(73) Assignees: Charite - Universitatsmedizin Berlin, Berlin (DE); Rheinische Friedrich-Wilhelms-Universitat Bonn, Bonn (DE)

(21) Appl. No.: 11/942,659

(22) Filed: Nov. 19, 2007

(30) Foreign Application Priority Data
Nov. 17, 2006 (DE) DE 102006054690.3

(51) Int. Cl. A61K 31/7068; C07F 17/00; A61P 35/00; A61P 31/00; A61P 37/00; A61P 29/00; A61K 31/28

(52) U.S. Cl. 514/49; 556/53; 514/492

(57) ABSTRACT

A medicament containing a compound of the following formulas (Ia) and (Ib):

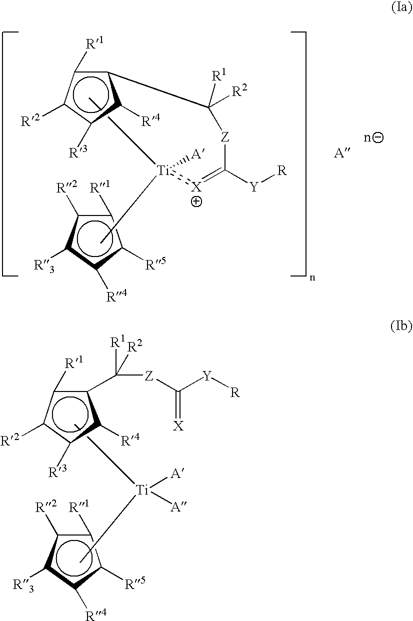

wherein
Z is selected from the group consisting of a covalent bond, at least one at least divalent hetero atom, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
R′¹ to R′⁴ and R″¹ to R″⁵ independently represent H, at least one hetero atom, especially oxygen, nitrogen or halogens, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
the two cyclopentadienyl rings are optionally linked together via any of residues R″¹ to R″⁵;
R¹ and R² independently represent H, a hetero atom, especially oxygen, nitrogen or halogens, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
A′, A″ is independently F, Cl, Br, I and/or a physiologically acceptable acid residue of an organic or inorganic acid;
n is a positive integer, especially 1, 2, 3;
X=O, S, NH, NR³ or NH⁴R⁵, wherein R³, R⁴ and R⁵ independently have the meanings as mentioned above for R′¹ to R′⁴;
Y is a covalent bond, O, S or NR⁶, wherein R⁶ has the meanings as mentioned above for R′¹ to R′⁴.

The present invention relates to substances and medicaments for the treatment of diseases caused by highly proliferating cells, and to methods for the preparation of such substances and medicaments.

Highly proliferating cells are the cause of various diseases including, for example, leukemias, malignant solid tumors as well as hyperproliferative diseases, such as psoriasis or keloid. Due to excessive proliferation, a disturbed balance arises between tissue regeneration on the one hand and the regulated death of cells from a tissue medium on the other hand. The natural homoeostasis is disturbed. This sensitive balance between tissue regeneration and degradation is regulated by the process of apoptosis. “Apoptosis” means the programmed cell death that every cell is able to perform. Particular signals intracellularly activate enzymes from the family of cysteine proteases, also referred to as caspases. This has the ultimate result that the cell dismantles itself from inside without causing inflammatory processes (Cohen, 1977). In this way, the excessive proliferation of cells and tissues is to be prevented.

In clinical practice, cytotoxic treatment methods, such as chemotherapy, radiation therapy and hyperthermia, are used to attempt to kill the excess cells, for example, tumor cells and to restore this disturbed balance for the treatment of diseases caused by excessively proliferating cells. The major part of chemotherapeutic agents achieve this by inducing apoptosis, the programmed cell death (Hannun, 1997). However, unfortunately it is found again and again that part of the malignant tumors early develop a resistance against radiation therapy or chemotherapy, or they are primarily refractory towards therapy (Hickman, 1996). Also, in part, the primary tumor and the metastases often respond quite differently to the respective therapy. Different defects in the apoptosis signal cascade could be identified as the cause of resistance development and primary resistance (Raisova et al., 2000). Due to such resistance, many tumor therapies remain unsatisfactory. This is true, in particular, of the therapy of recurrence of childhood acute lymphoblastic leukemia (ALL) in childhood. Despite of aggressive cytotoxic therapies, about 60% of the children afflicted recurrent ALL will die. Other tumor diseases in childhood and adulthood which are difficult to cure include, for example, mamma carcinoma, colon carcinoma, bronchial carcinoma, thyroid carcinoma, prostate carcinoma, testis cancer, lymphomas, leukemias as well as melanoma, neuroblastoma, osteosarcoma, Ewing sarcoma, nephroblastoma, rhabdomyosarcoma, teratoma, medulloblastoma, astrocytoma and glioblastoma. However, benign diseases are also treated by cytostatic medicaments, and it is still highly desirable that the therapeutic potency thereof be improved. This also applies to psoriasis, one of the most frequent benign diseases of the skin.

There is a strong interest in and great need for the development of substances and medicaments which can improve the percentage of successful healings and survival chances. Such substances should be highly selective against unnaturally proliferating resistant cells, especially for tumor diseases and leukemias, without too much damaging healthy cells.

Thus, the object of the invention is to provide novel medicaments that meet the above mentioned conditions. Another object is to provide substances which can be used according to the invention as a medicament for treating fast-proliferating cells that may be responsible for a pathological process.

According to the invention, this is achieved by the novel carbonyl-substituted titanocenes further described in the following and medicaments prepared therefrom.

Thus, the present invention relates to medicaments containing a compound of the following formulas (Ia) and (Ib):

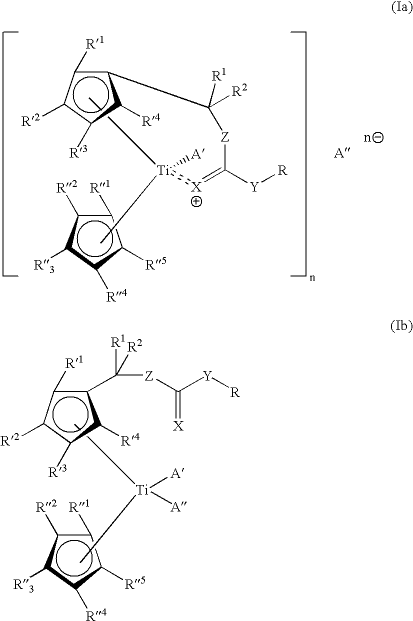

wherein
Z is selected from the group consisting of a covalent bond, at least one at least divalent hetero atom, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
R′¹ to R′⁴ and R″¹ to R″⁵ independently represent H, at least one hetero atom, especially oxygen, nitrogen or halogens, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
the two cyclopentadienyl rings are optionally linked together via any of residues R″¹ to R″⁵;
R¹ and R² independently represent H, a hetero atom, especially oxygen, nitrogen or halogens, a saturated, unsaturated, branched, unbranched and/or cyclic, substituted or unsubstituted hydrocarbon chain, a saturated or unsaturated, branched, unbranched and/or cyclic, substituted or unsubstituted hydrocarbon chain provided with hetero atoms in the chain thereof, or combinations thereof;
A′, A″ is independently F, Cl, Br, I and/or a physiologically acceptable acid residue of an organic or inorganic acid;
n is a positive integer, especially 1, 2, 3;
X=O, S, NH, NR³ or NH⁴R⁵, wherein R³, R⁴ and R⁵ independently have the meanings as mentioned above for R′¹ to R′⁴;
Y is a covalent bond, O, S or NR⁶, wherein R⁶ has the meanings as mentioned above for R′¹ to R′⁴;
R represents a saturated, unsaturated, branched, unbranched and/or cyclic, substituted or unsubstituted hydrocarbon chain provided or not with hetero atoms in the chain thereof, or combinations thereof.

Medicaments in which X=O can be employed, in particular.

Further, as the medicaments according to the invention, those are typically employed in which Y=NR⁶ and R⁶ has the meaning as mentioned above.

Further, according to the invention, medicaments are claimed in which X=O and Y=NR⁶, and R⁶ has the meaning as mentioned in the description of formula (Ia) or (Ib).

In particular, according to the invention, medicaments are employed in which Z=CR³R⁴, and wherein R³ and R⁴ as well as R′¹ to R′⁴ independently have the meanings as mentioned in the description of formula (Ia) or (Ib).

In the medicament according to the invention, A′ or A″ independently represent chloride and bromide, in particular.

In one embodiment of the medicament according to the invention, R′¹⁻⁴ may be a hydrogen atom. In another embodiment of the medicament according to the invention, R″¹⁻⁵ may be a hydrogen atom.

According to the invention, the substances having the structural formula (II)

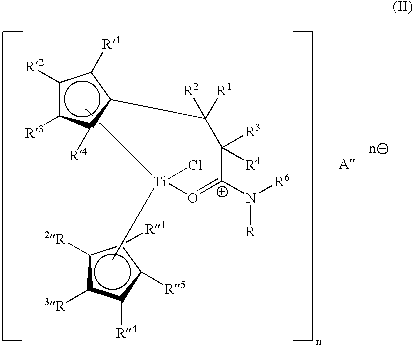

can be employed in the medicament according to the invention. In this formula, the residues and substituents have the same meanings as stated above in the description of formula (Ia) or (Ib).

In one embodiment of the medicament according to the invention in which the residues in the two cyclopentadienyl rings of the titanocene according to the invention are hydrogen each, it contains a compound of formula (III)

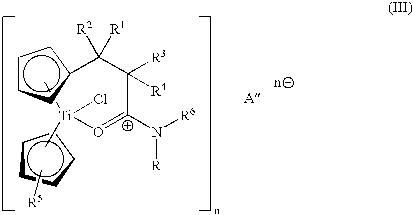

In this formula, the residues and substituents have the same meanings as stated above in the description of formula (Ia) or (Ib).

In another embodiment of the medicament according to the invention, it contains a compound of formulas (IVa) and (IVb):

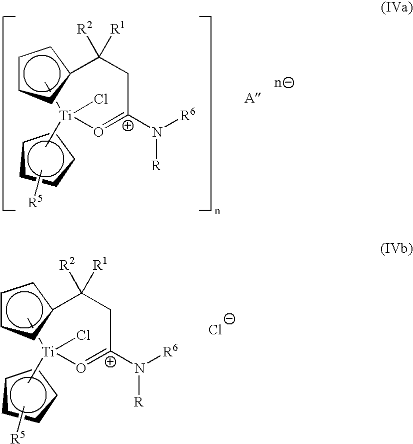

In these formulas, the residues and substituents have the same meanings as stated above in the description of formula (Ia) or (Ib).

In another embodiment of the medicament according to the invention, it contains a compound of formulas (Va), (Vb), (Vc) and (Vd):

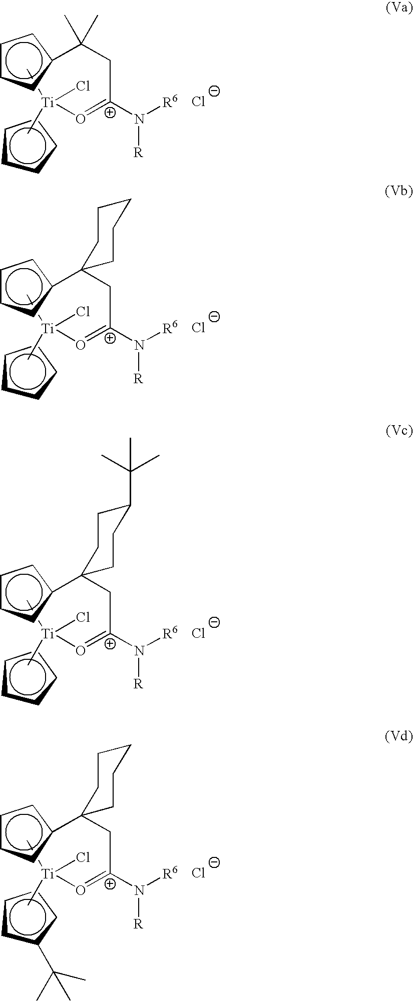

In these formulas, the residues and substituents have the same meanings as stated above in the description of formula (Ia) or (Ib).

In another embodiment of the medicament according to the invention, it contains a compound of formula (VI):

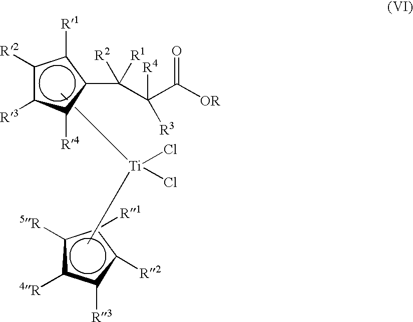

In this formula, the residues and substituents have the same meanings as stated above in the description of formula (Ia) or (Ib).

In another embodiment of the medicament according to the invention, it contains a compound of formulas (VIIa) and (VIIb):

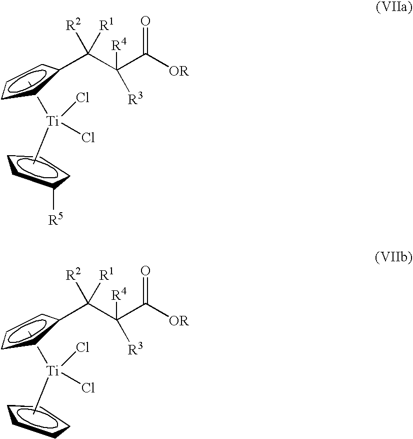

In these formulas, the residues and substituents have the same meanings as stated above in the description of formula (Ia) or (Ib).

In still another embodiment of the medicament according to the invention, it contains a compound of formulas (VIIIa) and (VIIIb):

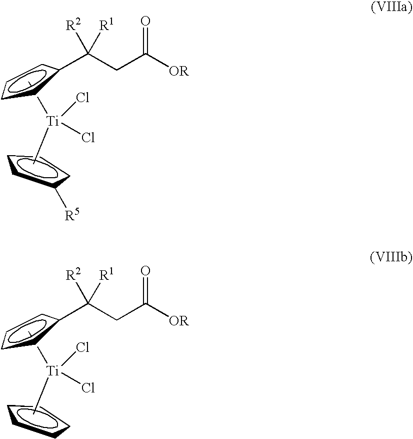

In these formulas, the residues and substituents have the same meanings as stated above in the description of formula (Ia) or (Ib).

In another embodiment of the medicament according to the invention, it contains a compound of formulas (IXa) and (IXb):

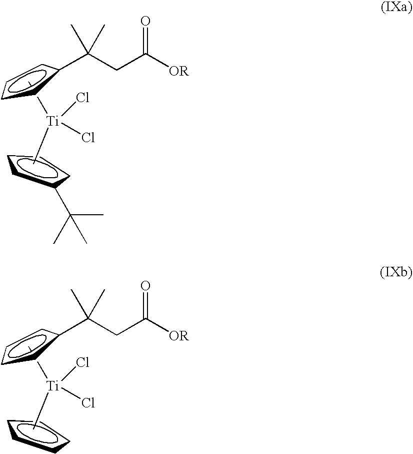

In these formulas, the residues and substituents have the same meanings as stated above in the description of formula (Ia) or (Ib).

In another embodiment of the medicament according to the invention, it contains ketone derivatives of the titanocene according to formula (X) shown below:

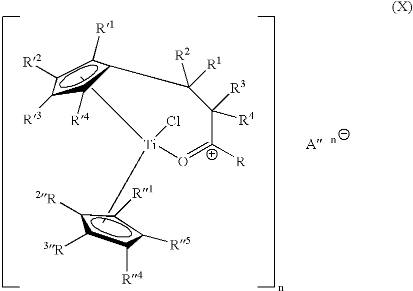

In this formula, the residues and substituents have the same meanings as stated above in the description of formula (Ia) or (Ib).

In another embodiment of the invention, this ketone derivative has the formula (XI):

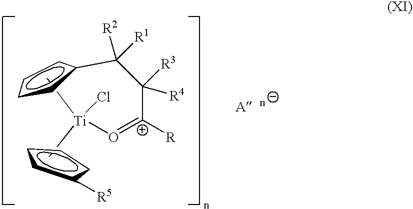

In this formula, the residues and substituents have the same meanings as stated above in the description of formula (Ia) or (Ib).

In still another embodiment of the invention, this ketone derivative has the formula (XII):

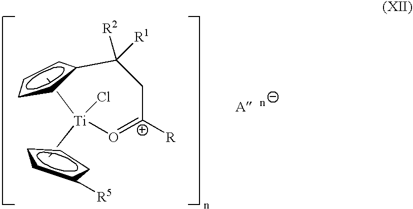

In this formula, the residues and substituents have the same meanings as stated above in the description of formula (Ia) or (Ib).

Further, the medicament according to the invention may contain a compound of formulas (XIIIa), (XIIIb), (XIIIc) and (XIIId):

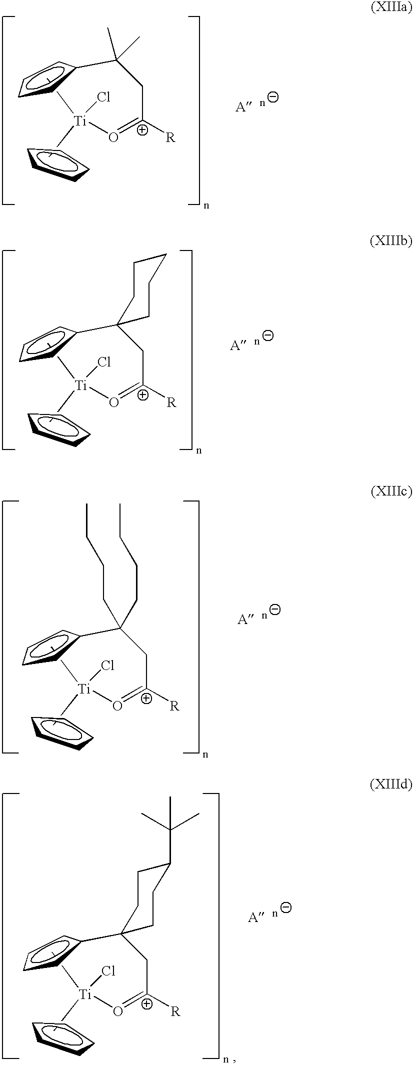

In these formulas, the residues and substituents have the same meanings as stated above in the description of formula (Ia) or (Ib).

In particular, the following compounds have proven useful by their high effectiveness in the medicament according to the invention, for example, for the treatment of leukemias:

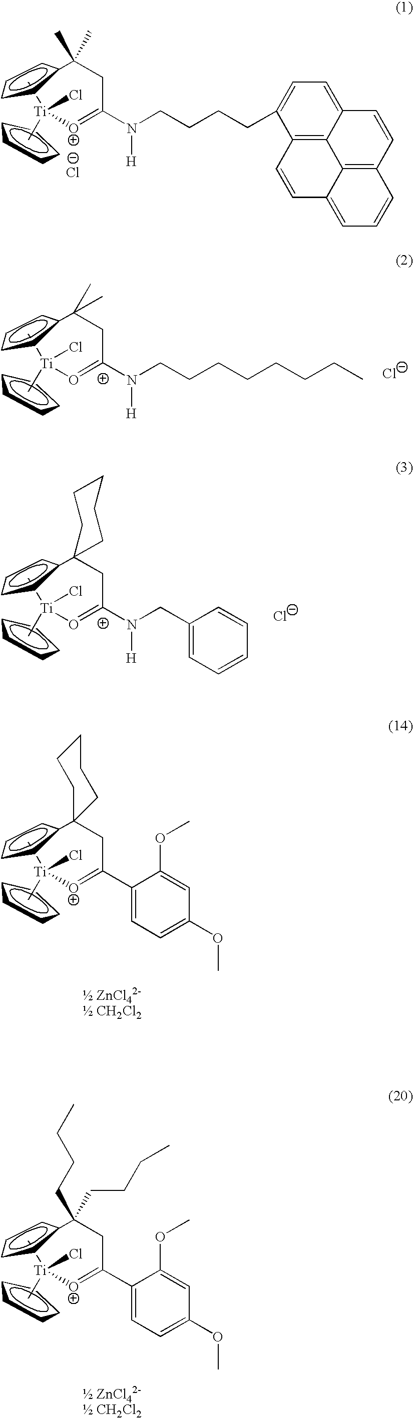

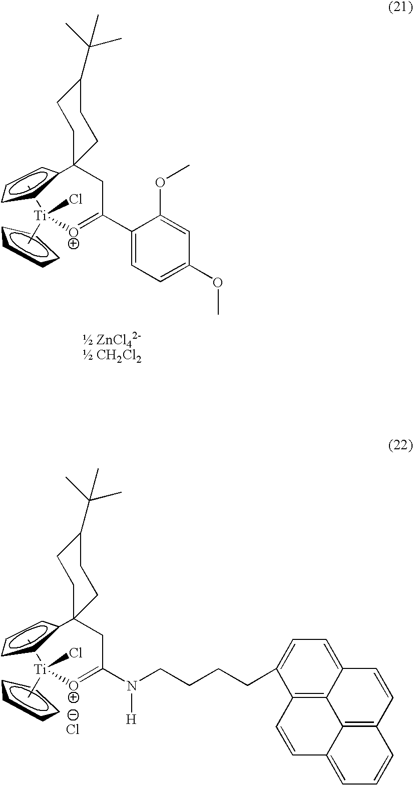

The present invention also provides compounds of structural formula (Ia) or (Ib), which are also claimed according to the invention, in which the substituents have the meanings as mentioned in connection with the description of formula (Ia) or (Ib), except for the compounds having the following structures:

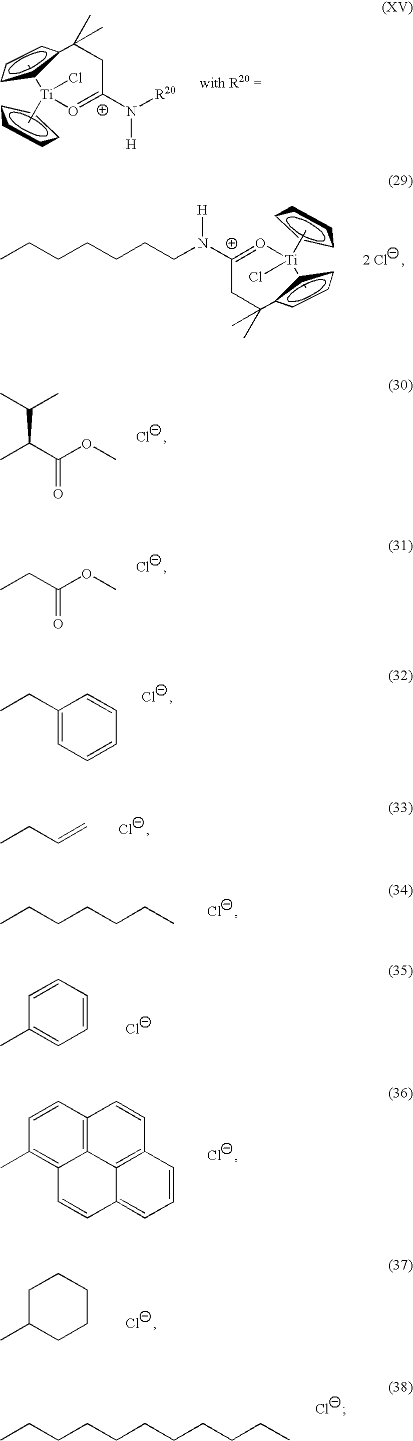

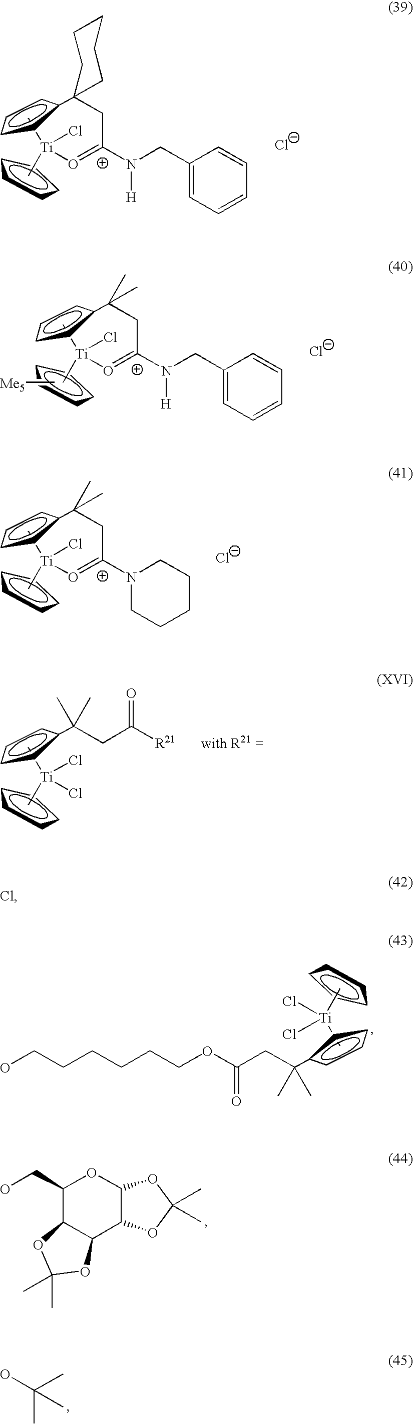

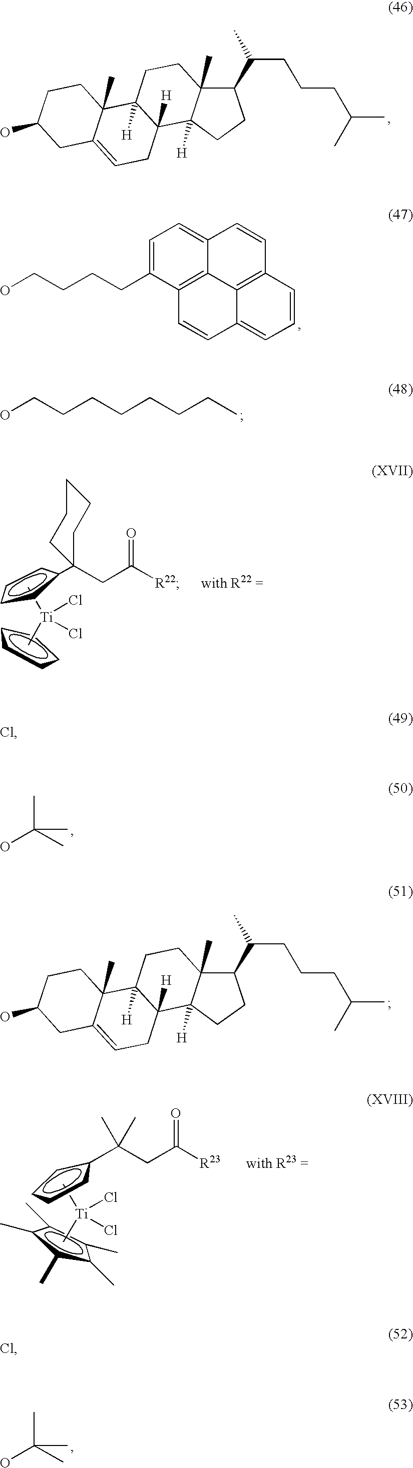

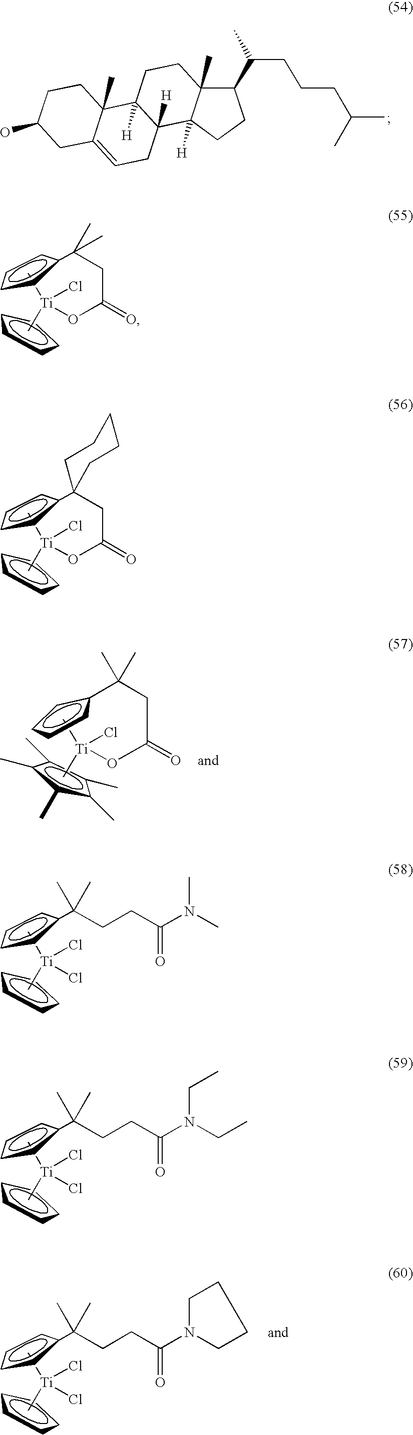

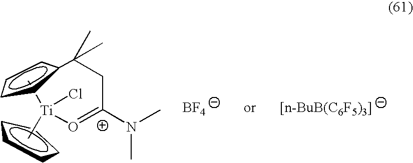

In one embodiment, the compound according to the invention has the following structural formula (II):

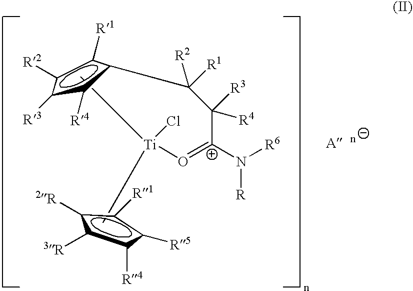

wherein the substituents have the meanings as mentioned in connection with the description of formula (Ia) or (Ib), and the disclaimer stated above is to be observed.

In one embodiment of the compound according to the invention in which the in which the residues in the two cyclopentadienyl rings of the titanocene according to the invention are hydrogen each, it has the structural formula (III)

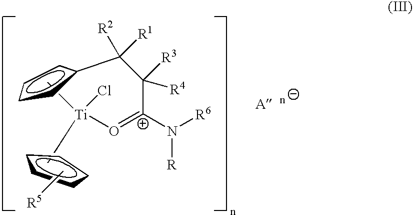

in which the substituents have the meanings as stated above, and the disclaimer stated above is to be observed.

In another embodiment, the compound according to the invention has the structural formulas (IV) and (IVa):

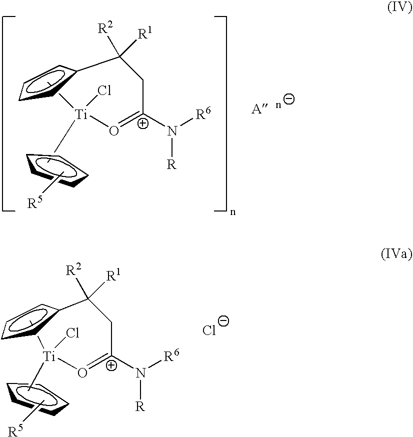

in which the substituents have the meanings as stated above, and the disclaimer stated above is to be observed.

In still another embodiment, the compound according to the invention has the structural formulas (Va), (Vb), (Vc) and (Vd):

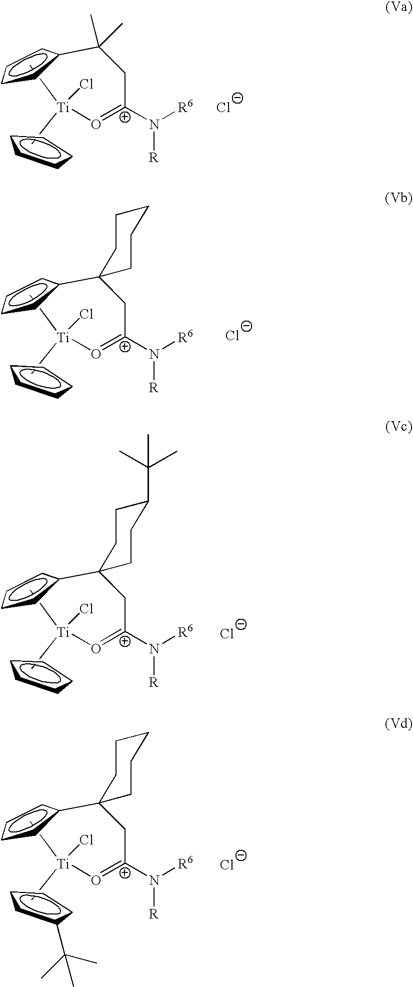

in which the substituents have the meanings as stated above, and the disclaimer stated above is to be observed.

In another embodiment, the compound according to the invention has the structural formula (VI):

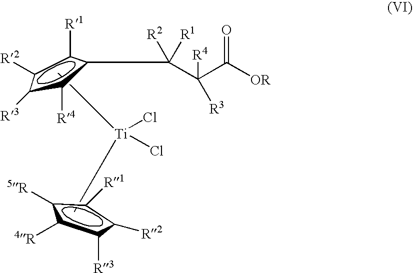

In this formula, the residues and substituents have the same meanings as stated above in the description of formula (Ia) or (Ib). The disclaimer stated above is also to be observed.

In another embodiment, the compound according to the invention has the structural formulas (VIIa) and (VIIb):

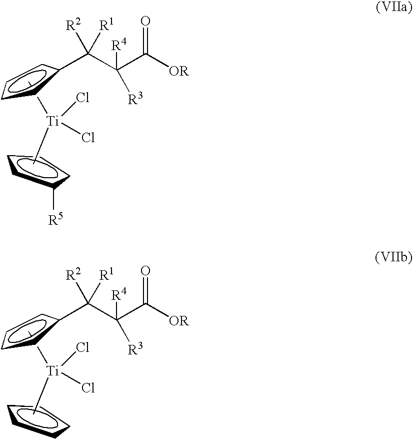

in which the substituents have the meanings as stated above, and the disclaimer stated above is to be observed.

In another embodiment, the compound according to the invention has the structural formulas (VIIIa) and (VIIIb):

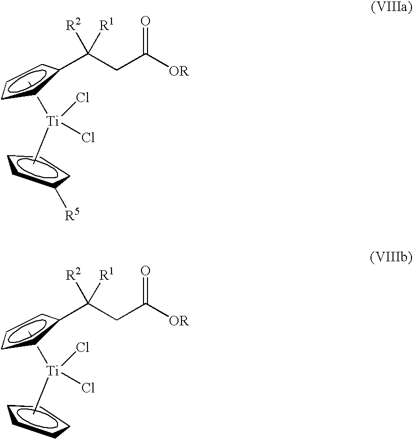

in which the substituents have the meanings as stated above, and the disclaimer stated above is to be observed.

In another embodiment, the compound according to the invention has the structural formulas (IXa) and (IXb):

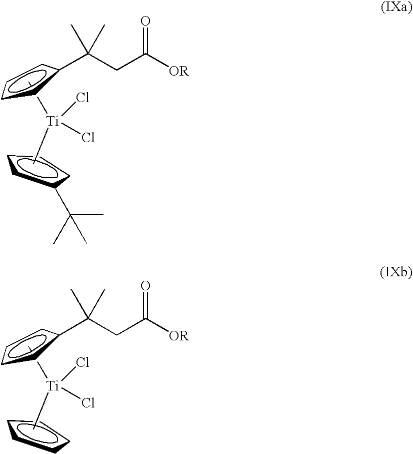

in which the substituents have the meanings as stated above, and the disclaimer stated above is to be observed.

In another embodiment, the compound according to the invention is a ketone derivatives of the titanocene and has the structural formula (X):

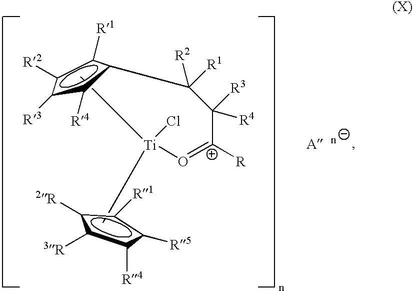

in which the substituents have the meanings as stated above, and the disclaimer stated above is to be observed.

In still another embodiment, the compound according to the invention is a ketone derivatives of the titanocene and has the structural formula (XI):

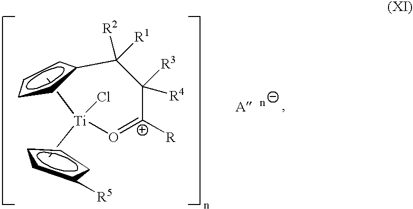

in which the substituents have the meanings as stated above, and the disclaimer stated above is to be observed.

In another embodiment, the compound according to the invention has the structural formula (XII):

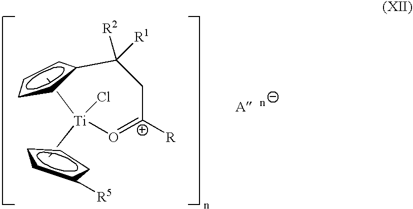

in which the substituents have the meanings as stated above, and the disclaimer stated above is to be observed.

In another embodiment, the ketone derivative of the titanocene is a compound having the following structural formula (XIIIa), (XIIIb), (XIIIc) and (XIIId)

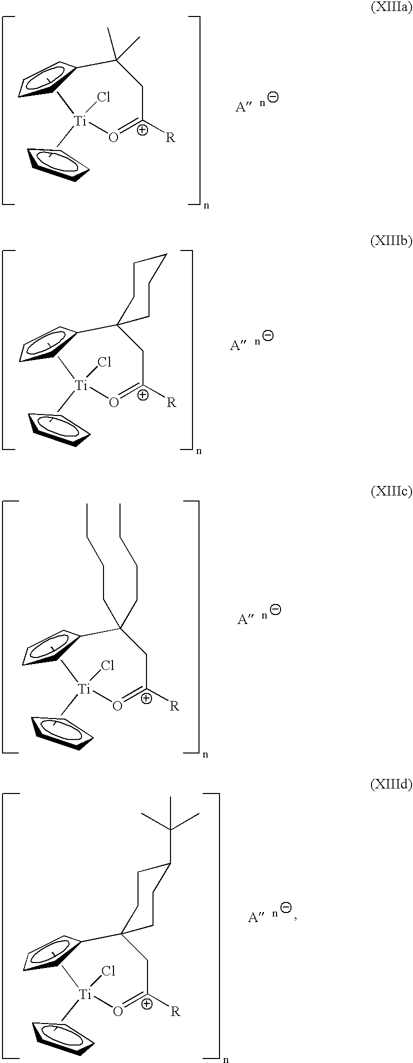

in which the substituents have the meanings as stated above, and the disclaimer stated above is to be observed.

The compounds employed in the medicament according to the invention and having the structural formulas (Ia) or (Ib) to (XIII) and the substituents described, which have the meanings as described above in connection with the description of formula (Ia) or (Ib), can be used according to the invention for the preparation of a medicament for the treatment of diseases related to fast-proliferating cells.

In particular, the diseases related to fast-proliferating cells that can be treated with the medicament according to the invention are selected from the group consisting of malignant diseases of the bone marrow, other hematopoietic organs, solid tumors, sarcomas, epithelial tumors, benign and semimalignant fast-proliferating tumors, skin diseases, such as psoriasis vulgaris, keloids, and also basaliomas, lymphomas, especially Hodgkin's and non-Hodgkin lymphomas, inflammatory, chronic inflammatory, bacterial and auto-immune diseases.

The use of the medicaments according to the invention for antibacterial, antimycotic, antiprotozoan, antiplasmodium, antiviral, antihelminthic or immunosuppressant therapies is also possible.

Further, the use of the medicaments according to the invention for the treatment of tumor diseases and leukemias, for the therapy of tumors of different origin, such as epithelial tumors, malignant diseases of the skin and for the therapy of malignant brain tumors, such as medulloblastoma, astrocytoma and/or glioblastoma is possible.

The titanocenes that can be employed in the medicament according to the invention are synthesized chemically and can be dissolved in organic or aqueous solvents. The medicament can be dissolved in an isotonic sodium chloride solution for i.v. applications or formulated as an ointment or oil (suspension) for external applications or applied as a suspension for oral applications.

The substances according to the invention are suitable for the therapy of pathological fast-proliferating tissue, especially bone marrow, but also of solid tumors, epithelial tumors and brain tumors. Also, a good applicability is seen in benign hyperproliferative diseases of the skin, such as psoriasis and keloid.

Without being limited by explanations of the mode of action of the compound according to the invention, the titanocenes seem to be particularly suitable for therapeutic purposes, since they exhibit a selective growth inhibition of highly proliferative cells because of apoptosis induction, and healthy cells are subjected to relatively little damage. The titanocenes are basically different from other cytostatic agents used in the prior art by being capable of breaking resistances against conventional cytostatic agents and leading allegedly resistant cells to death, because they possibly bind to a different site of the target cell.

The titanocenes are especially suitable for the treatment of tumor diseases and leukemias, but also for the therapy of tumors of different origin, such as epithelial tumors, malignant diseases of the skin and many more. Due to their relatively small size and the lipophilicity of the carbonyl-substituted titanocene molecules, they are able to trespass the blood-brain barrier, which also enables the therapy of malignant brain tumors, such as medulloblastoma, astrocytoma and glioblastoma.

It could be shown that the titanocenes have a high potency for apoptosis induction in different cell lines. Within the scope of apoptotic cell death, the cell is dismantled proteolytically by caspases from inside. Thereupon, the DNA is also fragmented, inter alia. This DNA fragmentation, which is specific for apoptosis, is considered a proof of apoptosis induction and is detected by means of flow cytometry on the single cell level.

BJAB cells (Burkitt lymphoma cell line) in different concentrations were treated with a titanocene derivative and incubated at 37° C. and 5% CO₂ for 72 hours. A control (untreated cell suspension) and a solvent control with dimethyl sulfoxide (DMSO) were continuously included. After incubation for 72 hours, apoptosis induction was detected by flow cytometry (FACS) through DNA fragmentation with propidium iodide (Eβmann, 2000).

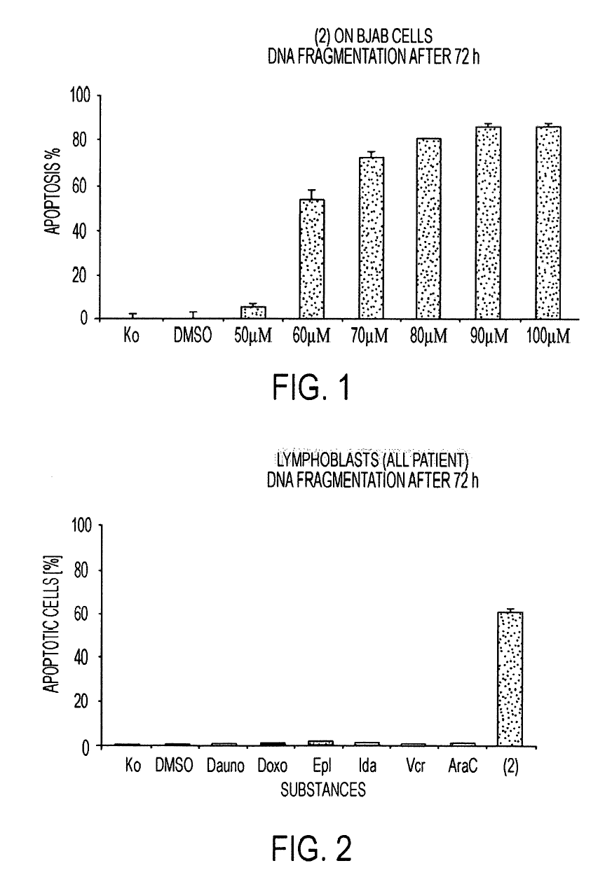

In FIG. 1, the titanocene derivative 4-[1-(dichlorotitanocenyl)]-4-methylbutyric acid n-octylamide shows a concentration-dependent apoptosis induction of up to 80%. The percentage indicates the proportion of apoptotic cells in the total population.

FIG. 1: BLAB mock cells (1×10⁵/ml) were treated with increasing concentrations of the titanocene derivative 4-[1-(dichlorotitanocenyl)]-4-methylbutyric acid n-octylamide (2) and incubated at 37° C. and 5% CO₂ for 72 h. After propidium iodide staining, the DNA fragments were detected by flow cytometry (FACS analysis). Kₒ (untreated cell suspension) and DMSO (solvent control) were included with equivalent treatment. A concentration-dependent apoptosis induction by 4-[1-(dichlorotitanocenyl)]-4-methylbutyric acid n-octylamide could be demonstrated. At a concentration of 100 μM 4-[1-(dichlorotitanocenyl)]-4-methylbutyric acid n-octylamide, apoptosis was induced in about 840% of the BJAB cells.

The apoptosis induction by titanocenes could be detected not only in permanent cell lines, but also in primary lymphoblasts ex vivo. After the primary cells have been obtained from ALL patients, the lymphoblasts were isolated at first and then treated with both commercially available cytostatic agents and the titanocene derivatives. The concentrations employed were selected to always be within the respective range of LD₅₀ when the BLAB cell line was used. Thereafter, the cells were incubated at 37° C. and 5% CO₂ for 60 hours, followed by staining with propidium iodide and quantifying by flow cytometry in FACS (Prokop et al., 2003). The carbonyl-substituted titanocenes impressively showed apoptosis induction in leukemia cells that were resistant against conventional cytostatic agents (FIG. 2). While conventional cytostatic agents did not have a significant effect, apoptosis could be induced by 4-[1-(dichlorotitanocenyl)]-4-methylbutyric acid n-octylamide (2) in about 60% of the leukemia cells ex vivo. Thus, 4-[1-(dichlorotitanocenyl)]-4-methylbutyric acid n-octylamide is capable of inducing programmed cell death in therapy-resistant tumor cells.

FIG. 2: Apoptosis induction by 4-[1-(dichlorotitanocenyl)]-4-methylbutyric acid n-octylamide (2) in primary lymphoblasts from an ALL patient after treatment of the isolated primary lymphoblasts with conventional cytostatic agents and 4-[1-(dichlorotitanocenyl)]-4-methylbutyric acid n-octylamide in the respective range of LD₅₀ in the BLAB cell line. The incubation was effected at 37° C. and 5% CO₂ for 60 h. Apoptosis induction was measured as DNA fragmentation by flow cytometry after propidium iodide staining. Conventional cytostatic agents (dauno-, doxo-, epi-, idarubicin, Vcr=vincristin, AraC=cytarabin) did not induce any significant apoptosis (K=control; EtOH and DMSO=solvent controls). After treatment with 4-[1-(dichlorotitanocenyl)]-4-methylbutyric acid n-octylamide, about 60% of the leukemia cells died though apoptosis induction.

In order to identify the whole range of activity of the titanocenes, studies on different cell lines were performed. Inter alia, an activity on leukemia cell lines (Nalm6, Reh), on cells of a hepatocellular carcinoma (HepG2) and on highly resistant, caspase-3-deficient mamma carcinoma cells (MCF-7) was found. This indicates that the titanocenes can be applied to malignant tumor diseases of different entities.

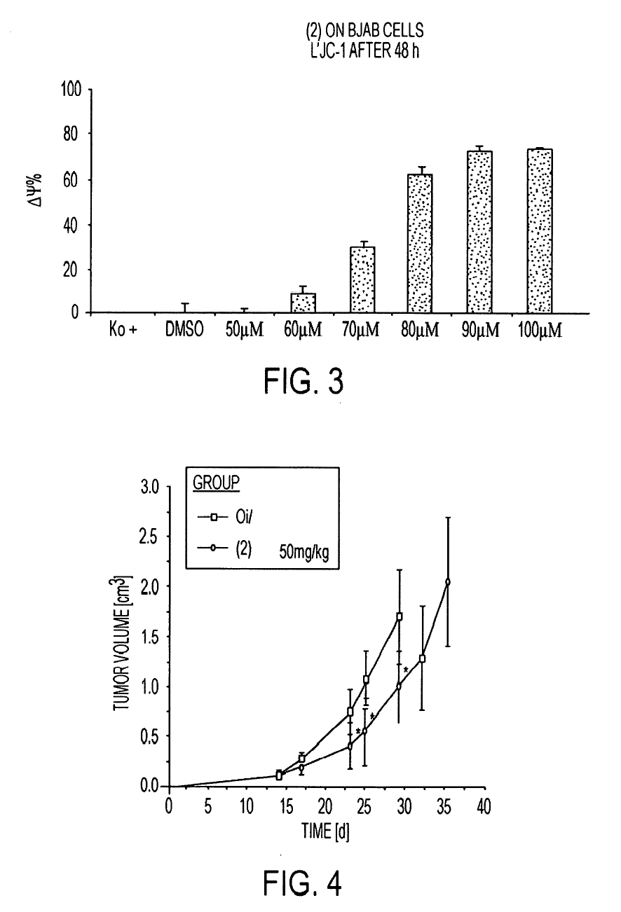

Mechanistic examinations with the mitochondria-specific stain JC-1 (Wieder et al., 2001) showed that the apoptosis signal cascade induced by the carbonyl-substituted titanocenes is mediated by mitochondria. After treatment of the cells with different concentrations of a titanocene including a zero control and a solvent control (DMSO), the cells are incubated at 37° C. and 5% CO₂ for 48 hours. It was found that the proportion of cells having a lowered mitochondrial membrane potential (ΔΨₘ) increases as the concentration of titanocene increases (FIG. 3). This indicates an activation of the mitochondria during the apoptotic process (Diller et al. 2005).

FIG. 3: Concentration-dependent change of the mitochondrial membrane potential in BJAB cells upon treatment with 4-[1-(dichlorotitanocenyl)]-4-methylbutyric acid n-octylamide. The incubation was effected at 37° C. and 5% CO2 for 48 h including zero and solvent controls, followed by staining the cells with the mitochondria-specific stain JC-1 and detection of the color change and thus the change of the mitochondrial membrane potential by flow cytometry. A concentration-dependent change of the mitochondrial membrane potential (ΔΨₘ) induced by 4-[1-(dichlorotitanocenyl)]-4-methylbutyric acid n-octylamide was found in more than 70% of the cells.

FIG. 4: In first in vivo experiments after oral therapy, it could be shown that the carbonyl-substituted titanocenes inhibit the growth of lymphoma cells in mice.

After an oral therapy with a microcrystalline suspension of (2) (50 mg/kg body weight), the growth of human lymphoma cells in SCID mice (n=6, control: n=10) was significantly inhibited (Mann-Whitney U test: p<0.05). The mean values of the tumor volumes (*=significance) with standard deviations as a function of therapy duration (application of (2) from day 14 with 5×50 mg/kg body weight per week p.o. over 2 weeks) are shown. A good tolerability of the active substance was shown.

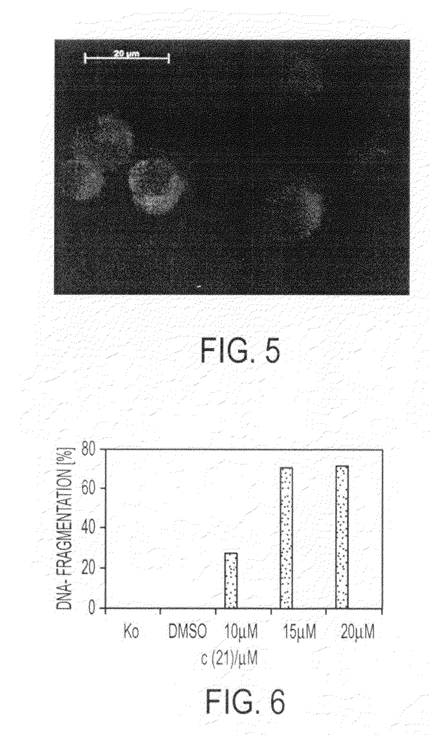

FIG. 5: After incubation with the titanocene (22) for 24 h, fluorescence-microscopic examinations on lymphoma cells (BLAB) show enrichment of the substance in compartments of the cytoplasm. Therefore, carbonyl-substituted titanocenes are also suitable for the labeling and diagnostic detection of fast-proliferating cells and thus for their diagnostic application.

Since the activity of the cytostatic agents develops in the malignant cells through a specific apoptosis induction, it is reasonable to measure this effect and thus the selectivity of our compounds rather than stating, as usual, the non-specific cytotoxicity described by the LC₅₀ values. Therefore, for examining the structure-effect relationship of our titanocenes, we have established the concentration (AC₅₀) at which a specific apoptosis is induced in 500% of the lymphoma cells. Since only the desired selective effect is covered, the AC₅₀ is higher than the corresponding LC₅₀ value.

The effectiveness of some compounds is summarized in Table 1.

TABLE 1
Activity of the carbonyl-substituted titanocenes against the BJAB cell line

| | | | | | | | | | |
|---|---|---|---|---|---|---|---|---|---|
| Complex | (8) | (14) | (21) | (20) | (26) | (62) | (2) | (1) | (22) |
| AC₅₀ [μM] | 100 | 50 | 12.5 | 35 | 80 | >100 | 55 | 50 | 30 |

The AC₅₀ of the most active compound to date (21) in lymphoma cells (BJAB) is 12.5 μM (FIG. 4). For determining this value, lymphoma cells (10⁵/ml BJAB cells) were incubated in different concentrations with (21) at 5% CO₂ and 37° C. for 72 h. After staining with propidium iodide, the DNA fragmentation was detected by flow cytometry by means of a FACS analysis. A concentration-dependent apoptosis induction of above 74% of the cell population was found. Only 3% of the cells were necrotic.

FIG. 6 shows the apoptosis induction by the complex (21).

A non-specific cytotoxic effect of the titanocenes via necrosis could be excluded by measuring the extracellular lactate dehydrogenase (LDH) release by ELISA reader detection after incubation for 3 h. These results show that the cytostatic effect of the carbonyl-substituted titanocenes is selectively mediated by apoptosis induction. The non-specific damage from (2) is so low that the viability of the cell population is above 94% even for the highest concentration.

The cellular changes typical of apoptosis are also found in the microscopic examination at 72 h after treatment with titanocenes (FIG. 6).

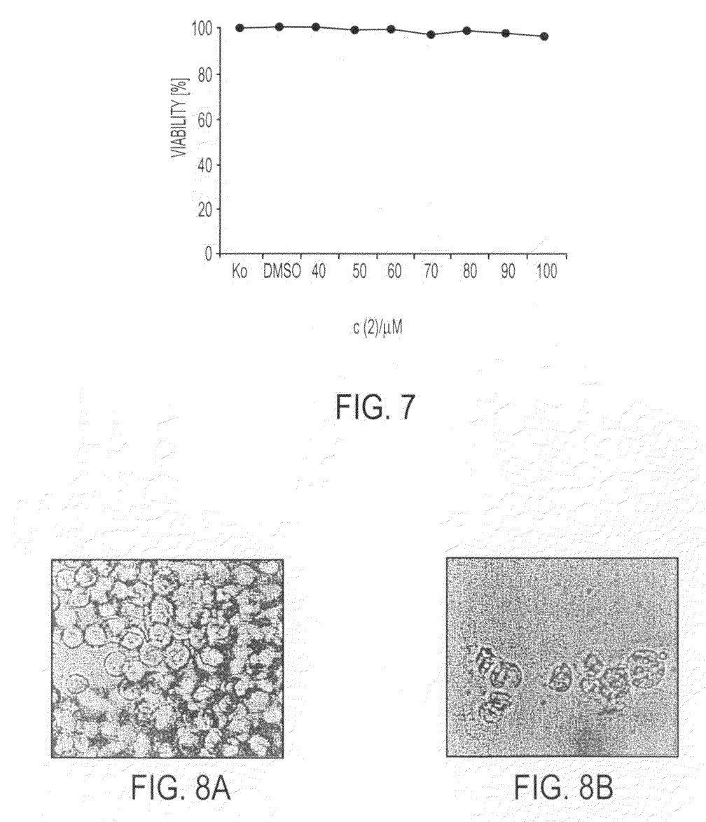

FIG. 7 shows the exclusion of non-specific cytotoxic damage from (2) by measuring the cellular release of lactate dehydrogenase (LDH release) with ELISA technology.

FIG. 8 shows a microscopic view of apoptosis induction in BJAB cells by (2). FIG. 8A represents the zero control after 72 h (incubation: intact lymphoma cells in dense colonies), and FIG. 8B represents the apoptosis induction after incubation with (2) (75 μM). In addition to some fragments of cells that had been dead already, only singular lymphoma cells that are clearly changed morphologically can be seen with a significant swelling and “blebbing” that is characteristic of apoptosis.

These results are supported by in vivo data that demonstrate a good tolerability of the titanocenes up to a concentration of 75 mg/kg body weight.

The data shown in Table 1 indicate a dependence of the biological effectiveness of the titanocenes according to the invention on their structure. Since ester-substituted complexes and (629 are not effective, the cationic coordination at the titanium is essential. The introduction of sterically demanding substituents on the “lower” cyclopentadienyl ring results in a significantly reduced activity. For a broad screening, it is of great importance that the most effective cationic amide- and ketone-substituted complexes have clearly different substituents on the carbonyl group. While in the ketone compounds according to the invention synthesized in an exemplary manner, the 2,4-dimethoxyphenyl residue results in a very high effectiveness, all aniline and benzyl amine derivatives of the amides examined to date are inactive. Only the introduction of long alkyl chains, optionally with terminal aryl substitution, results in titanocenes having a high apoptosis induction.

The gem-dimethyl group adjacent to the cyclopentadienyl ligand in the complexes (8), (2) and (1) proved not to be ideal. The activities observed of these complexes against the BJAB cell line are interesting, but by no means sufficient. Surprisingly, the introduction of the cyclohexyl residue in 2b already causes a significant increase of the activity of our compounds. This effect could be increased even more by introducing the 4-tert-butylcyclohexyl substituent. The complexes (21) and (22) are among the complexes with the highest effectiveness described in the literature to date. The complex (20) according to the invention with a di-n-butyl substituent is between (22) and (21) in terms of effectiveness. Thus, in order to achieve a high biological activity, a group that is as sterically demanding as possible and non-polar adjacent to the cyclopentadienyl ligand can be advantageous.

Thus, in summary, the carbonyl-substituted titanocenes according to the invention represent a novel class of active substances showing an extraordinarily high apoptosis induction in a wide variety of tumor and leukemia cells. The complexes can thus be employed against a broad range of malignant diseases. In addition, the invention provides a general design principle for biologically active titanocenes. In vivo experiments additionally show a significant inhibition of tumor growth in SCID mice with human lymphomas.

Thus, the present invention also relates to a diagnostic agent comprising a compound of general formulas (Ia) and/or (Ib).

Further, the present invention also relates to a combination of the medicaments and compounds according to the invention with cytostatic agents, especially nucleoside analogues, such as cytarabin (AraC).

In lymphoma cells (BLAB cells), it could be shown that carbonyl-substituted titanocenes in combination with conventional cytostatic agents, such as nucleoside analogues, can synergistically induce apoptosis.

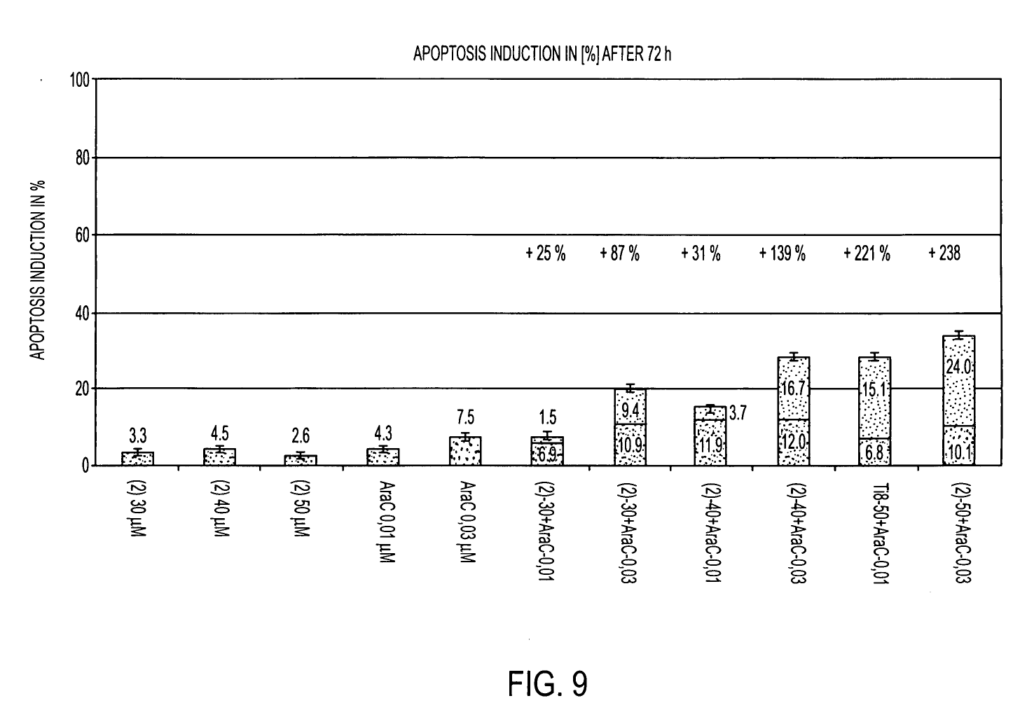

FIG. 9 shows the corresponding effects. Lymphoma cells (BLAB) were treated with the carbonyl-substituted titanocene (2), the conventional nucleoside analogue cytarabin (AraC), which is used in the therapy of malignant diseases, and with the combination of (2) and AraC in various concentrations for 72 h. The apoptosis induction was examined by flow cytometry by measuring the DNA fragmentation after staining the cells with propidium iodide. The measured values of three independent examinations are shown, wherein the error bars show the standard deviations from the mean. A significant synergistic effect of (2) and AraC could be observed with respect to apoptosis induction.

Thus, it is shown that carbonyl-substituted titanocenes can significantly enhance or improve the anti-tumor activity of conventional cytostatic agents, such as nucleoside analogues.

Another great advantage of the antileukemic/antitumor effect of the titanocenes resides in the relatively low non-specific cytotoxicity of these active substances.

The carbonyl-substituted titanocenes show relatively low non-specific cytotoxic effects while the apoptosis induction is pronounced. This could be shown by measuring the hardly detectable release of lactate dehydrogenase (LDH) (Schlawe et al., 2004) in BLAB cells after treatment with titanocenes over a period of 3 h.

The present invention also relates to a process for the preparation of the compounds according to the invention.

Key intermediates of the synthesis of the carbonyl-substituted titanocenes are the cyclic carboxylates C and the carboxylic acid chloride-substituted titanocenes D. The preparation of the carbonyl-substituted titanocenes from D is shown below.

The synthesis of the carbonyl-substituted titanocenes is based on the extraordinarily high reactivity of titanocene-substituted carboxylic acid chlorides D towards nucleophiles (NuH), which are commercially available in a very wide variety or can be prepared in a simple manner. The titanocene-substituted carboxylic acid chlorides D behave like organic carboxylic acid chlorides. Therefore, virtually any carbonyl-substituted titanocene can be prepared in this way. This is shown below in an exemplary manner for amides, esters and ketones.

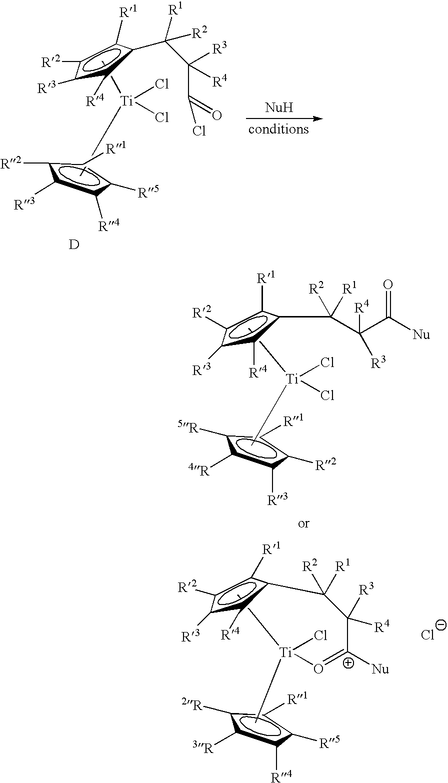

The preparation of the carboxylic acid chlorides D is effected quantitatively from cyclic carboxylates C, which are available in a short and simple sequence from commercially available or readily obtainable substrates according to a protocol by Gansäuer (Gansäuer 2005).

The modular preparation of the cyclic carboxylates C and their reaction with SOCl₂ to form D is shown below.

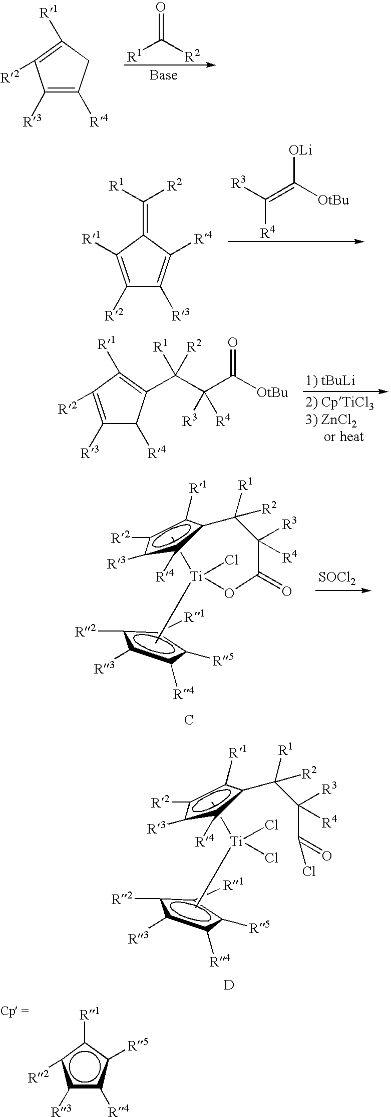

The substituents and residues have the same meanings as stated in the description of formula (Ia) or (Ib).

The necessary ester-substituted cyclopentadienes are prepared in a two-step synthesis from cyclopentadienes, ketones and the enolate of tert-butyl acetate. At first, a fulvene is formed in virtually quantitative yield, to which an ester enolate is added. In these steps, no chromatographic purification is necessary. After deprotonation, metallization with a cyclopentadienyltitanium trichloride yields the titanocene shown, which is converted to the cyclic carboxylate C by treatment with ZnCl₂ or by mere heating. All the shown residues R¹′-R⁴′ and R¹″-R⁵″ and R¹-R⁴ can thus be introduced in a short and extraordinarily efficient sequence.

The preparation of the compounds according to the invention is conveniently effected according to the following reaction scheme. In an exemplary manner, the preparation starts from the cyclic carboxylate C, which is then converted to the ester (VIIIb) according to synthesis route A and to the amide (IVb) according to synthesis route B.

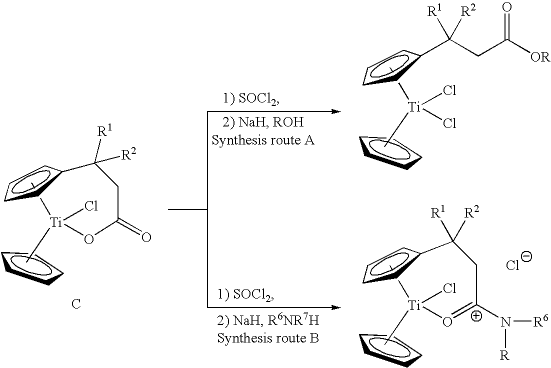

The preparation of the corresponding ketone-substituted titanocenes also starts from the cyclic carboxylate C, following reaction route C.

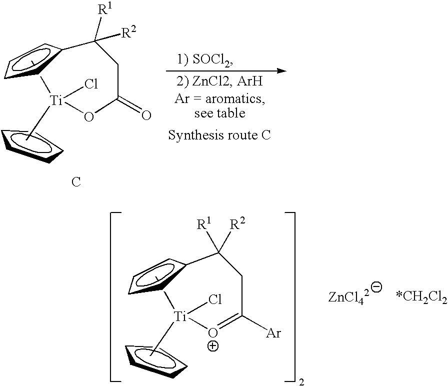

The invention is further illustrated with reference to the following Examples.

### EXAMPLE 1

### Preparation of the Ester- and Amide-Substituted Titanocenes

To a suspension of titanocene carboxylate (10.0 mmol) in CH₂Cl₂ (10 mL), SOCl₍3 mL) is added, and the mixture is stirred at room temperature for 3 h. The solvent and the excess of SOCl₂ were removed under high vacuum. The residue was dissolved in CH₂Cl₂ (15 mL) and added dropwise to a mixture of NaH (720 mg, 30.0 mmol) and the nucleophile (alcohol or amine) (10.0 mmol) in (10 mL) and stirred for 16 h. After filtration over Celite, the solvent is removed under reduced pressure.

The mixture is taken up in toluene (30 ml), filtered over Celite, and the residue is dissolved in CH₂Cl₂ (20 mL) and washed with HCl (1 M, admixed with 1 g NaCl per 10 mL) (3×200 mL). The organic phase is dried (MgSO₄), and the solvent is removed under reduced pressure. If desired, the solid obtained may also by recrystallized from toluene or CH₂Cl₂.

| | | |
|---|---|---|
| Carboxylate C | Nucleophile | Yield, Product |

| | | |
|---|---|---|
| R¹, R² = CH₃ | n-C₈H₁₇NH₂ | 39%, R¹, R² = CH₃, R⁶ = n-C₈H₁₇, |
| | | R = H |
| R¹, R² = (CH₂)₅ | n-C₈H₁₇NH₂ | 98%, R¹, R² = (CH₂)₅, R⁶ = n-C₈H₁₇, |
| | | R = H |
| R¹, R² = (CH₂)₅ | H₂NCH₂CO₂CH₃ | 68%, R¹, R² = (CH₂)_{5,} |
| | | R⁶ = CH₂CO₂CH₃, R = H |
| R¹, R² = CH3 | H₂NCH(CH₂Ph)CO₂CH₃ | 31%, R¹, R² = CH_{3,} |
| | | R⁶ = CH(CH₂Ph)CO₂CH₃, R = H |
| R¹, R² = CH3 | H₂NCH(CH₂CH₂SCH₃)CO₂CH₃ | 24%, R¹, R² = CH₃, R⁶ = |
| | | CH(CH₂CH₂SCH₃)CO₂CH₃ , R = H |
| R¹, R² = CH3 | Menthol | 72%, R¹, R² =CH₃, R = Menthyl |

### EXAMPLE 2

### Preparation of the Ketone-Substituted Titanocenes

To a suspension of titanocene carboxylate (1 mmol), SOCl₂ (3 mL) is added, and the mixture is stirred at room temperature for 3 h. The excess of SOCl₂ is removed under high vacuum. The product is dissolved in 5 ml of CH₂Cl₂, ZnCl₂ (0.680 g, 5 mmol) is added and stirred for 16 h. After the addition of further (10 mL), the mixture is washed with HCl (1 N, 1 g NaCl per 10 mL). For purification, the product is precipitated from the CH₂Cl₂ solution by adding cyclohexane, and filtered off. After repeating this precipitation five times, the product is obtained as an orange solid.

| ArH | Yield, Ar |
|---|---|
| 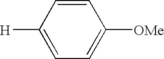 | 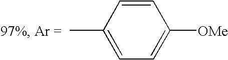 |
| 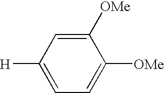 | 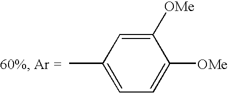 |
| 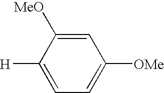 | 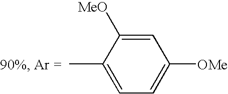 |

### Spectroscopic Data for the Compounds Prepared

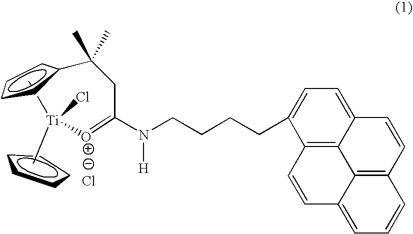

M.p.: 144-146° C.

¹H-NMR (400 MHz, CDCl₃) δ=12.20-12.32 (br., 1H); 7.84-8.21 (m, 9H); 6.60-6.70 (br., 1H); 6.39-6.48 (br., 2H); 6.15-6.33 (br., 5H); 5.72-5.80 (br., 1H); 3.34-3.48 (m, 2H); 3.11-3.31 (m, 2H); 1.83-2.03 (m, 2H); 1.66-1.80 (m, 2H); 1.15-1.23 (m, 3H); 1.02-1.14 (m, 2H).

¹³C-NMR (100 MHz, CDCl₃) δ=176.0; 149.3; 136.2; 131.2; 130.8; 129.7; 128.5; 127.5; 127.4; 126.6; 125.9; 124.9; 124.9; 124.8; 123.5; 121.3; 119.6; 118.8; 117.6; 109.4; 53.5; 41.8; 34.2; 32.9; 30.1; 29.1; 28.7; 27.8; 26.5.

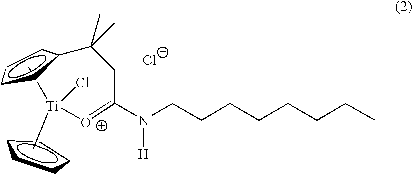

Melting point: 68° C. (decomposition)

¹H-NMR (400 MHz, CDCl₃): δ=11.82 (dd, J=5.6 Hz, 5.6 Hz, 1H), 7.10-7.15 (m, 1H, H), 7.00-7.05 (m, 1H), 6.68 (s, 5H), 6.54-6.58 (m, 1H), 5.90-5.94 (m, 1H), 3.12-3.30 (m, 2H), 3.31 (d, J=14.0 Hz, 1H), 3.03 (d, J=14.0 Hz, 1H), 1.55-1.65 (m, 2H), 1.20-1.32 (m, 10H), 1.28 (s, 3H), 1.27 (s, 3H).

¹³C-NMR (100 MHz, CDCl₃): δ=175.4, 149, 124.8, 120.9, 119, 117.7, 108.9, 45.5, 41.2, 33.8, 31.2, 29.2, 28.5, 28.3, 26.6, 26.3, 22.0, 13.6.

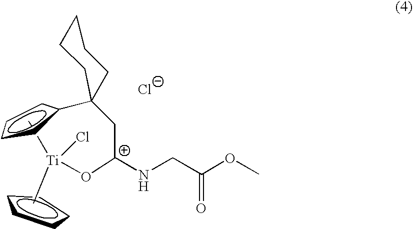

M.p.: 228° C. (decomposition)

¹H-NMR (400 MHz, CDCl₃): δ=12.42 (dd, ³J_{HH}=5.1 Hz, 1H), 7.21-7.17 (m, 1H), 7.08 (brs, 1H), 6.66 (s, 5H), 6.57 (brs, 1H), 6.10 (brs, 1H), 3.96 (d, ³J_{HH}=5.7 Hz, 2H), 3.72 (s, 3H), 3.39 (d, ³J_{HH}=13.5 Hz, 1H), 2.82 (d, ³J_{HH}=13.5 Hz, 1H), 1.98-1.85 (m, 2H), 1.80-1.63 (m, 3H), 1.60-1.49 (m, 1H), 1.44-1.31 (brs, 3H), 1.20-1.07 (brs, 1H).

¹³C-NMR (100 MHz, CDCl₃, RT): δ=178.31, 167.91, 150.33, 125.63, 121.90, 121.36, 115.78, 111.35, 52.78, 46.64, 42.35, 38.73, 38.51, 34.12, 25.35, 22.24, 21.84.

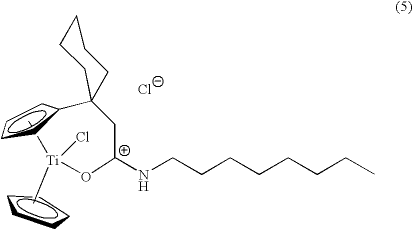

M.p.: 87° C. (decomposition)

¹H-NMR (400 MHz, CDCl₃): δ=11.87 (brs, 1H), 7.13 (brs, 1H), 7.06 (brs, 1H), 6.68 (s, 5H), 6.53 (brs, 1H), 3.38 (d, ³J_{HH}=8.8 Hz, 1H), 3.24 (t, ³J_{HH}=5.7 Hz, 1H), 3.17 (t, ³J_{HH}=5.7 Hz, 1H), 2.81 (brs, 1H), 1.89-1.17 (m, 22H), 0.84 (t, ³J_{HH}=6.8 Hz, 3H).

¹³C-NMR (100 MHz, CDCl₃, RT):

δ=175.92, 150.58, 125.34, 121.11, 119.52, 116.20, 110.33, 41.74, 38.71, 38.32, 34.09, 31.84, 29.24, 29.19, 28.85, 27.10, 25.41, 22.70, 22.19, 21.73, 14.18.

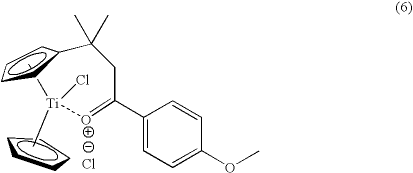

M.p.: 115° C. (decomposition)

¹H-NMR (400 MHz, CDCl₃) 6=(400 MHz, CDCl₃) δ=8.19 (d, ³J=8.2 Hz, 1H); 7.63 (s, 1H); 7.43 (s, 1H); 7.06 (d, ³J=8.6 Hz, 1H); 6.96 (s, 5H); 6.42 (s, 1H); 6.02 (s, 1H); 4.12 (d, ³J=13.7 Hz, 1H); 3.94 (s, 3H); 3.22 (d, ³J=14.4 Hz, 1H); 1.57 (s, 3H); 1.00 (s, 3H).

¹³C-NMR (100 MHz, CDCl₃, RT) δ=211.9; 168.5; 150.7; 138.0; 136.2; 127.1; 123.8; 122.8; 122.4; 119.1; 117.6; 115.8; 111.9; 56.7; 52.3; 31.2; 25.8; 21.6.

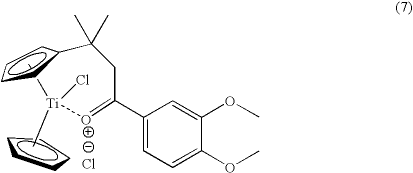

M.p.: 134° C. (decomposition)

¹H-NMR (400 MHz, CDCl₃) δ=8.02 (d, ³J=9.4 Hz, 1H); 7.66 (s, 1H); 7.52 (s, 1H); 7.36 (s, 1H); 7.08 (d, ³J=7.3 Hz, 1H); 6.96 (s, 5H); 6.44 (s, 1H); 6.02 (s, 1H); 4.15 (d, ³J=13.6 Hz, 1H); 4.01 (s, 3H); 3.98 (s, 3H); 3.32 (d, ³J=12.5 Hz, 1H); 1.60 (s, 3H); 1.00 (s, 3H).

¹³C-NMR (100 MHz, CDCl₃, RT) δ=212.2; 158.6; 150.7; 149.6; 137.9; 129.1; 128.3; 127.3; 122.7; 122.3; 117.7; 112.2; 111.9; 57.1; 56.5; 52.3; 31.1; 26.9; 25.8.

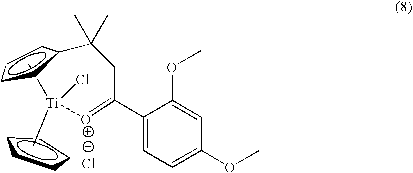

M.p.: 84° C. (decomposition)

¹H-NMR (400 MHz, CDCl₃) δ=7.89 (d, ³J=8.9 Hz, 1H); 7.65 (s, 1H); 7.47 (s, 1H); 6.94 (s, 5H); 6.65 (d, ³J=9.9 Hz, 1H); 6.52 (s, 1H); 6.35 (s, 1H); 6.01 (s, 1H); 4.18 (d, ³J=13.8 Hz, 1H); 4.05 (s, 3H); 3.98 (s, 3H); 3.11 (d, ³J=13.8 Hz, 1H); 1.50 (s, 3H); 1.04 (s, 3H).

¹³C-NMR (100 MHz, CDCl₃, RT) δ=209.7; 170.5; 166.0; 150.5; 138.0; 125.4; 123.4; 122.1; 120.0; 117.4; 112.1; 108.9; 98.7; 57.2; 56.9; 54.8; 36.8; 31.2; 25.8.

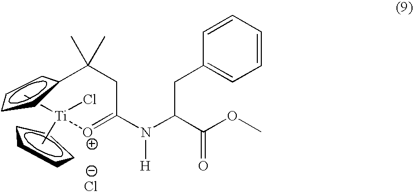

M.p.: 73-75° C.

¹H-NMR (400 MHz, CDCl₃) δ=12.35 (d, ³J=8.2 Hz; 1H)*; 12.27 (d, ³J=7.7 Hz, 1H)*; 7.36-7.47 (m, 2H); 7.25-7.35 (m, 2H); 7.18-7.24 (m, 1H); 7.08-7.15 (m, 1H); 6.72 (s, 5H)*; 6.50 (s, 5H)*; 6.23 (s, 1H); 6.00 (s, 1H)*; 5.95 (s, 1H)*; 4.63 (dd, ³J=14.8 Hz, ³J=8.2 Hz, 1H)*; 4.48 (dd, ³J=13.9 Hz, ³J=6.7 Hz, 1H)*; 3.75 (s, 3H)*; 3.73 (s, 3H)*; 3.30-3.47 (M, 2H); 3.25 (d, ³J=13.3 Hz, 1H); 2.93 (d, ³J=13.6 Hz, 1H); 1.28 (s, 3H); 1.23 (s, 3H).

¹³C-NMR (100 MHz, CDCl₃, RT) δ=177.3; 169.8*; 169.6*; 151.3*; 150.9*; 138.8*; 136.8*; 136.2*; 129.6*; 129.4*; 128.8*; 128.7*; 127.2*; 127.0*; 124.2*; 121.4*; 120.9*; 119.6*; 116.6*; 110.6*; 109.9*; 56.7*; 56.1*; 53.0*; 52.8*; 47.0*; 36.2*; 35.8*; 34.8*; 34.5*; 30.3*; 30.2*; 25.6*; 24.5*.

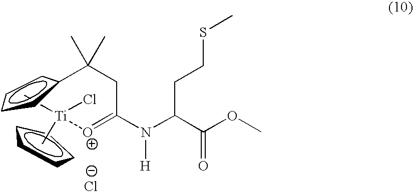

M.p.: 60° C. (decomposition)

¹H-NMR (400 MHz, CDCl₃) δ=12.15 (d, ³J=5.16 Hz, 1H)*; 12.05 (s, 1H)*; 7.19 (m, 1H); 7.00-7.18 (m, 1H); 6.70 (s, 5H)*; 6.68 (s, 5H)*; 6.60 (s, 1H); (s, 1H)*; 5.94 (br., 1H); 4.50-4.59 (br., 1H)*; 4.42-4.50 (br., 1H)*; 3.76 (s, 3H)*; 3.70 (s, 3H)*; 2.94-3.07 (m, 2H)*; 2.78-2.91 (m, 2H)*); 2.60-2.78 (m, 2H); 2.33-2.45 (m, 1H); 2.13-2.26 (m, 1H); 2.06 (s, 3H); 1.37 (s, 3H)*; 1.35 (s, 3H)*; 1.31 (s, 3H)*; 1.28 (s, 3H).

¹³C-NMR (100 MHz, CDCl₃, RT) δ=178.2*; 178.0*; 170.5*; 170.2*; 150.8*; 150.2*; 125.4*; 125.2*; 121.6; 119.9*; 119.6*; 117.3*; 116.9*; 110.3*; 109.8*; 53.4*; 53.3*; 53.1*; 53.0*; 46.6*; 46.2*; 35.0*; 34.7*; 30.6*; 30.5*; 30.4*; 29.9*; 28.8*; 28.4*; 26.2*; 25.7*; 15.2*; 15.0*.

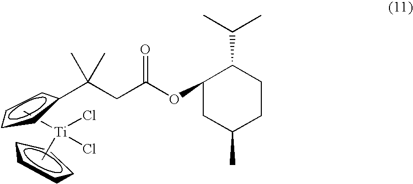

M.p.: 155-157° C.

¹H-NMR (400 MHz, CDCl₃) δ=6.62 (s, 2H); 6.56 (s, 5H); 6.46 (dd, ³J=6.4 Hz, ³J=2.8 Hz, 2H); 4.57 (ddd, ³J=11.0 Hz, ³J=10.9 Hz, ³J=4.2 Hz, 1H); 2.55 (dd, ³J=18.4 Hz, ³J=13.6 Hz, 2H); 1.79 (d, ³J=2.6 Hz, 1H); 1.76 (dddd, ³J=13.8 Hz, ³J=7.0 Hz, ³J=7.0 Hz, ³J=2.7 Hz, 1H); 1.59-1.69 (m, 2H); 1.48 (s, 3H); 1.47 (s, 3H); 1.35-1.45 (m, 1H); 1.22-1.34 (m, 1H); 0.92-1.06 (m, 1H); 0.76-0.92 (m, 9H); 0.69 (d, ³J=6.9 Hz, 3H).

¹³C-NMR (100 MHz, CDCl₃, RT) δ=170.8; 146.6; 120.7; 120.6; 120.3; 117.5; 117.1; 74.3; 49.5; 46.9; 41.0; 36.8; 34.3; 31.4; 27.9; 27.7; 26.2; 23.3; 22.1; 20.8; 16.3.

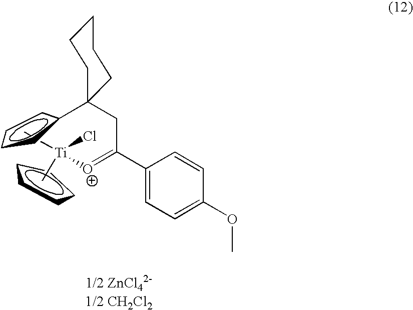

M.p.: 150° C. (decomposition)

¹H-NMR (400 MHz, CDCl₃) δ=8.12 (d, ³J=6.6 Hz, 2H); 7.67 (m, 1H); 7.23 (m, 1H); 6.97 (s, 5H); 6.96 (d, ³J=8.9 Hz, 1H); 6.38 (m, 1H); 6.17 (m, 1H); 4.31 (m, 1H); 3.94 (s, 3H); 3.10 (m, 1H); 2.23 (m, 1H); 2.03 (m, 1H); 1.23-1.45 (m, 6H); 1.02-1.14 (m, 2H),

¹³C-NMR (100 MHz, CDCl₃) δ=212.4; 168.3; 149.9; 136.1; 127.6; 124.6; 123.7; 122.3; 116.5; 115.6; 112.3; 56.7; 41.6; 39.5; 33.9; 27.0; 25.3; 22.9; 22.2.

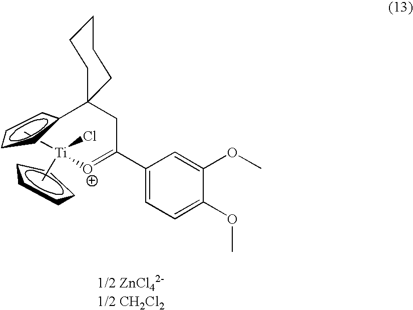

M.p.: 153° C.

¹H-NMR (400 MHz, CDCl₃) δ=8.05-8.20 (br., 1H); 7.52-7.50 (m, 2H); 7.40-7.50 (br., 1H); 7.11-7.22 (br. 1H); 6.85-7.05 (br., 5H); 6.27-6.35 (br., 1H); 6.10-6.20 (br., 1H); 4.25-4.45 (br., 1H); 3.99 (s, 3H); 3.92 (s, 3H); 2.91-3.10 (br., 1H); 2.69-2.89 (br., 1H); 2.09-2.20 (br., 1H); 1.90-2.05 (br., 1H); 1.16-1.36 (m, 6H); 0.88-1.03 (br., 1H).

¹³C-NMR (100 MHz, CDCl₃) δ=212.7; 158.4; 149.9; 149.5; 127.8; 124.6; 123.7; 122.3; 116.5; 112.3; 112.1; 57.1; 56.5; 41.7; 39.4; 34.0; 26.9; 25.3; 22.8; 22.2.

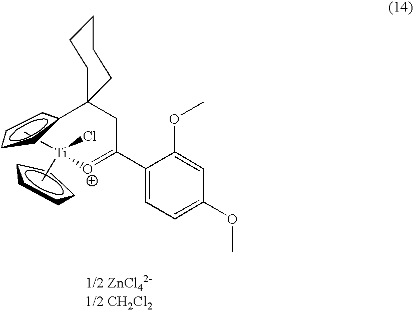

M.p.: 145° C. (decomposition)

¹H-NMR (400 MHz, CDCl₃) δ=7.95 (d, ³J=9.2 Hz, 1H); 7.66-7.22 (m, 1H); 7.30-7.37 (m, 1H); 6.94 (s, 5H); 6.64 (dd, ³J=9.1 Hz, ⁴J=2.2 Hz, 1H); 6.47 (d, 1H); 6.31-6.36 (m, 1H); 6.12-6.17 (m, 1H); 4.43 (d, ²J=14.2 Hz, 1H); 4.03 (s, 3H); 3.96 (s, 3H); 2.96 (d, ²J=14.2 Hz, 1H); 2.18-2.28 (m, 1H); 1.86-1.96 (m, 1H); 1.57-1.66 (m, 1H); 1.47-1.55 (m, 1H); 1.26-1.41 (m; 5H); 1.04-1.16 (m, 1H).

¹³C-NMR (100 MHz, CDCl₃) δ=209.4; 170.6; 166.0; 149.5; 137.2; 124.0; 123.5; 121.8; 117.4; 116.4; 112.2; 109.3; 98.6; 57.5; 57.2; 53.6; 41.6; 39.2; 33.9; 29.5; 25.5; 22.8; 22.2; 21.8.

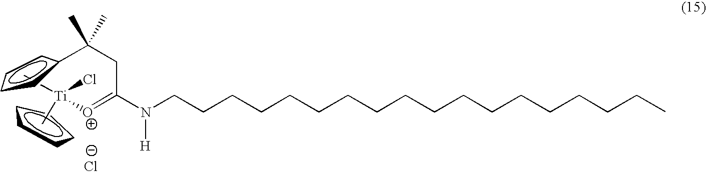

¹H-NMR (400 MHz, CDCl₃) δ=12.39-12.48 (br., 1H); 6.91-6.98 (br., 1H); 6.56-6.68 (br., 7H); 6.00-6.05 (br., 1H); 3.35-3.44 (m; 1H); 3.18-3.32 (m, 1H); 1.61-1.70 (m, 2H); 1.56 (s, 3H); 1.34 (s, 3H); 1.19-132 (m, 32H); 0.88 (t, ³J=7.0 Hz, 3H).

¹³C-NMR (100 MHz, CDCl₃) δ=176.0; 150.3; 125.2; 121.3; 119.4; 117.3; 109.5; 46.4; 41.8; 34.5; 32.0; 30.3; 29.7; 29.7; 29.6; 29.6; 29.4; 29.3; 28.9; 27.1; 26.2; 22.7; 14.2.

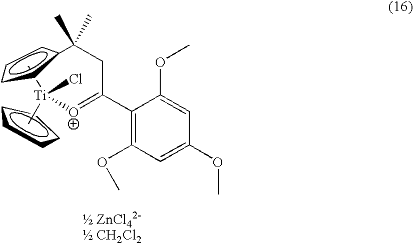

M.p.: 128-130° C.

¹H-NMR (400 MHz, CDCl₃) δ=7.60-7.70 (m, 1H); 7.48-7.59 (m, 1H); 6.89 (s, 5H); 6.30-6.38 (m, 1H); 6.10-6.20 (m, 2H); 5.99-6.08 (m, 1H); 3.98 (s, 3H); 3.93 (s, 6H); 3.78 (d, ³J=14.5 Hz, 1H); 2.97 (d, ³J=14.4 Hz, 1H); 1.43 (s, 3H); 1.03 (s, 3H).

¹³C-NMR (100 MHz, CDCl₃) δ=211.7; 169.3; 164.1; 149.7; 123.4; 122.3; 121.9; 117.5; 116.9; 112.7; 110.3; 91.4; 58.2; 57.1; 56.9; 53.6; 37.1; 31.0; 26.0.

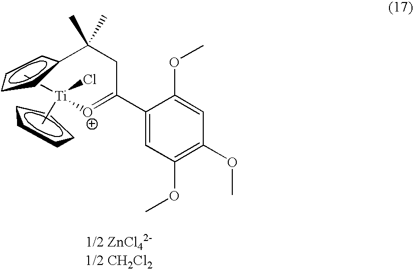

M.p.: 165° C. (decomposition)

¹H-NMR (400 MHz, CDCl₃) δ=6.89-6.92 (m, 2H); 6.47-6.49 (m, 1H); 6.37 (s, 5H); 6.18-6.21 (m, 1H); 6.15-6.17 (m, 1H); 5.71-5.74 (m, 1H); 4.07-4.09 (m, 1H); 4.04-4.06 (m, 1H); 3.90 (s, 3H); 3.88 (s, 3H); 3.83 (s, 3H); 1.38 (s, 3H); 1.29 (s, 3H).

¹³C-NMR (100 MHz, DMSO) δ=207.6; 162.7; 161.5; 157.9; 150.3; 148.5; 121.9; 119.8; 117.1; 113.0; 111.8; 109.7; 98.1; 57.8; 57.5; 56.3; 55.3; 35.3; 34.1; 33.1.

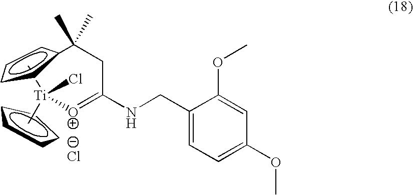

M.p.: 122° C.

¹H-NMR (400 MHz, CDCl₃, RT): δ=12.58 (brs, 1H), 7.29 (s, 1H), 6.88 (brs, 1H), 6.57 (brs, 1H), 6.47 (s, 5H), 6.45-6.37 (m, 3H), 6.04 (brs, 1H), 4.48 (d, ²J=14.7 Hz, 1H), 4.33 (d, ²J=13.6 Hz, 1H), 3.87 (s, 3H), 3.78 (s, 3H), 3.44 (d, ²J=13.5 Hz, 1H), 2.87 (d, ²J=13.1 Hz, 1H), 1.28 (s, 3H), 1.24 (s, 3 H).

¹³C-NMR (75 MHz, CDCl₃): δ=175.93, 160.69, 157.93, 150.11, 129.86, 124.61, 121.25, 120.27, 116.86, 116.59, 109.61, 104.23, 98.36, 55.51, 55.43, 46.43, 39.72, 34.46, 29.35, 26.57.

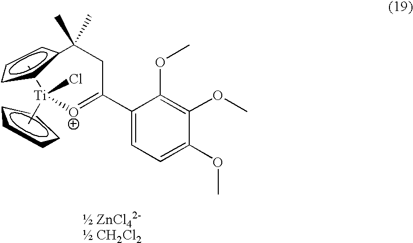

M.p.: 135° C.

¹H-NMR (400 MHz, CDCl₃): δ=7.92 (d, ³J=8.9 Hz, 1H), 7.57 (s, 2H), 6.99 (s, 1H), 6.96 (s, 5H), 6.37 (brs, 1H), 7.57 (brs, 1H), 4.19 (d, ²J=14.5 Hz, 1H), 4.04 (s, 3H), 3.99 (s, 3H), 3.81 (s, 3H), 3.15 (d, ²J=14.4 Hz, 1H), 1.48 (s, 3H), 1.03 (s, 3H).

¹³C-NMR (75 MHz, CDCl₃): δ=212.1, 162.9, 157.5, 150.2, 142.5, 132.2, 123.6, 122.5, 122.4, 122.1, 117.7, 111.9, 108.86, 63.0, 61.1, 57.2, 55.0, 36.8, 31.2, 25.6.

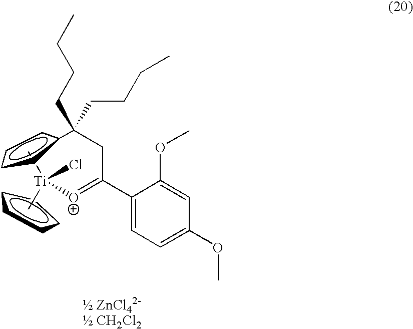

M.p.: 110° C. (decomposition)

¹H-NMR (400 MHz, CDCl₃): δ=7.89 (d, ³J=9.2 Hz, 1H), 7.60 (brs, 1H), 7.46 (brs, 1H), 6.92 (s, 5H), 6.66-6.48 (m, 2H), 6.38 (brs, 1H), 5.98 (brs, 1H), (brs, 1H), 4.06 (s, 3H), 3.99 (s, 3H), 2.90 (brs, 1H), 1.99-0.54 (m, 18H).

¹³C-NMR (75 MHz, CDCl₃, RT): δ=209.2, 170.4, 165.8, 150.6, 136.9, 124.6, 122.1, 121.2, 117.8, 117.5, 114.0, 111.4, 109.0, 98.5, 57.5, 57.2, 43.5, 38.1, 32.5, 27.4, 26.1, 24.9, 22.9, 22.8, 14.2, 13.8.

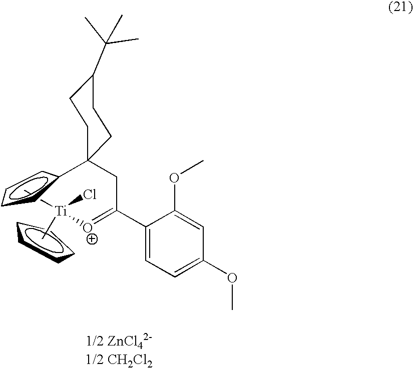

M.p.: 175° C. (decomposition)

¹H-NMR (400 MHz, CDCl₃): δ=7.88 (d, ³J=9.1 Hz, 1H), 7.60 (brs, 1H), 7.47 (brs, 1H), 6.92 (s, 5H), 6.62 (d, ³J=9.1 Hz, 1H), 6.55 (s, 1H), 6.25 (brs, 1H), 6.19 (brs, 1H), 4.21 (d, ²J=13.4 Hz, 1H), 4.05 (s, 3H), 3.98 (s, 3H), 2.84 (d, ²J=13.4 Hz, 1H), 1.78-0.81 (m, 9H), 0.63 (s, 9H).

¹³C-NMR (75 MHz, CDCl₃): δ=209.8, 170.5, 165.9, 148.5, 137.3, 124.4, 123.8, 121.8, 117.5, 116.1, 112.5, 109.1, 98.6, 58.2, 57.4, 57.2, 47.3, 42.2, 40.5, 34.2, 32.3, 27.4, 24.1, 23.4.

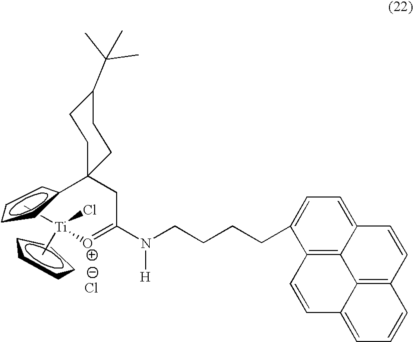

M.p.: 195° C.

¹H-NMR (400 MHz, CDCl₃): δ=12.50-12.60 (br., 1H); 7.85-8.35 (m, 9H); 6.51 (d, ³J=3.15 Hz, 1H); 6.29-6.32 (br., 1H); 5.99 (s, 5H); 5.97-6.00 (br., 1H); 5.58-5.62 (br., 1H); 3.43 (t, ³J=6.2 Hz, 2H); 3.04-3.32 (m, 3H); 2.99 (d, ²J=13.0 Hz, 1H); 2.50 (d, ²J=12.6 Hz, 1H); 0.71 (s, 9H); 0.35-2.08 (m, 12H).

¹³C-NMR (75 MHz, CDCl₃): δ=176.3; 146.9; 136.5; 131.4; 130.8; 129.8; 128.7; 127.7; 127.6; 127.4; 127.0; 126.7; 126.0; 125.0; 124.9; 124.9; 123.7; 122.7; 120.7; 119.4; 119.2; 115.8; 110.8; 53.5; 49.6; 46.9; 41.6; 38.3; 35.4; 32.6; 32.1; 29.3; 28.5; 27.3; 23.2; 16.8.

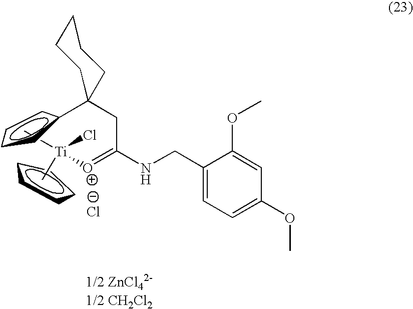

M.p.: 98° C.

¹H-NMR (300 MHz, CDCl₃): δ=12.04 (s, 1H), 7.15 (d, ³J=6.2 Hz, 2H), 6.85 (brs, 1H), 6.52 (s, 5H), 6.41 (brs, 1H), 6.39-6.30 (m, 2H), 6.01 (brs, 1H), 4.34 (dd, ²J=14.2 Hz, ³J=4.0 Hz, 1H), 4.22 (dd, ²J=14.4 Hz, ³J=3.3 Hz, 1H), 3.77 (s, 3H), 3.70 (s, 3H), 3.36 (d, ²J=11.0 Hz, 1H), 2.74 (d, ²J=13.0 Hz, 1H), 1.80-1.39 (m, 6H), 1.32-0.99 (m, 4H).

¹³C-NMR (75 MHz, CDCl₃): δ=175.8, 160.7, 157.9, 149.5, 129.9, 125.4, 121.1, 119.5, 116.7, 116.2, 110.6, 104.1, 98.2, 55.4, 55.4, 46.0, 39.8, 38.2, 37.6, 34.7, 25.3, 22.0, 21.7.

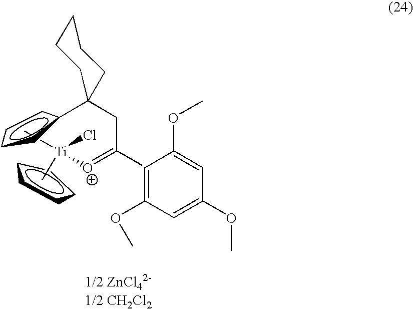

M.p.: 161° C.

¹H-NMR (400 MHz, CDCl₃): δ=7.72 (brs, 1H), 7.39 (brs, 1H), 6.91 (s, 5H), 6.30 (brs, 1H), 6.21 (s, 1H), 6.08 (s, 2H), 4.00 (d, ²J=12.5 Hz, 1H), 3.93 (s, 6H), 3.92 (s, 3H), 2.89 (d, ²J=12.6 Hz, 1H), 1.84-1.07 (m, 10H).

¹³C-NMR (100 MHz, CDCl₃): δ=212.3, 169.2, 163.8, 148.3, 124.3, 123.8, 121.7, 116.2, 113.0, 110.8, 91.4, 59.1, 57.2, 56.9, 42.1, 39.1, 34.3, 25.5, 22.9, 22.3.

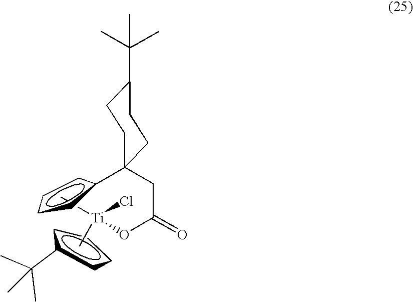

M.P.: >220° C.

¹H-NMR (400 MHz, CDCl₃): δ=6.85 (dd, ³J=5.2 Hz, ³J=3.0 Hz, 1H); 6.65 (dd, ³J=5.3 Hz, ³J=2.3 Hz, 1H); 6.58 (dd, ³J=5.3 Hz, ³J=2.3 Hz, 1H); 6.52 (dd, ³J=5.2 Hz, ³J=3.0 Hz, 1H); 6.31 (dd, ³J=5.4 Hz, ³J=3.0 Hz, 1H); 6.27 (dd, ³J=5.2 Hz, ³J_{HH}=2.3 Hz, 1H); 5.90 (dd, ³J=5.6 Hz, ³J=3.1 Hz, 1H); 5.88 (dd, ³J=5.3 Hz, ³J=2.4 Hz, 1H); 2.70 (d, ³J=12.2 Hz, 1H); 2.24 (ddd, ³J=13.8 Hz, ³J=5.9 Hz, ³J=2.9 Hz, 1H); 2.15 (d, ³J=12.2 Hz, 1H); 2.01 (ddd, ³J=13.5 Hz, ³J=6.2 Hz, ³J=3.1 Hz, 1H); 1.53 (d, ³J=3.4 Hz, 1H); 1.49 (d, ³J=3.4 Hz, 1H); 1.45-1.48 (m, 1H); 1.29-1.32 (m, 1H); 1.26 (s, 9H); 0.8-1.05 (m, 3H); 0.73 (s, 9H).

¹³C-NMR (75 MHz, CDCl₃): δ=171.7; 152.5; 143.5; 142.6; 129.8; 129.7; 129.5; 129.5; 121.0; 116.4; 113.7; 110.6; 51.5; 47.5; 38.6; 38.0; 34.6; 32.4; 30.7; 27.5; 24.3; 23.4; 23.3.

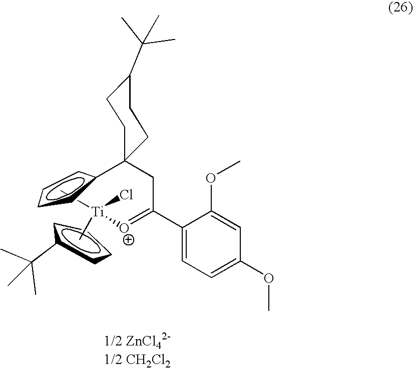

M.p.: 167° C. (decomposition)

¹H-NMR (400 MHz, CDCl₃): δ=7.84 (d, ³J_{HH}=9.2 Hz, 1H); 7.65-7.74 (br., 1H); 7.33-7.40 (br., 1H); 7.09 (d, ³J_{HH}=2.1 Hz, 1H); 6.98-7.05 (br., 2H); 6.62-6.70 (br., 1H); 6.55-6.62 (br., 2H); 6.15 (d, ³J_{HH}=6.2 Hz, 2H); 4.19 (d, ³J_{HH}=13.0 Hz, 1H); 4.0 (s, 3H); 3.99 (s, 3H); 2.72 (d, ³J_{HH}=13.0 Hz, 1H); 2.66 (d, ³J_{HH}=12.6 Hz, 1H); 1.68 (t, ³J_{HH}=13.5 Hz, 1H); 1.43-1.58 (m, 3H); 1.20-1.33 (m, 2H); 1.16 (s, 9H); 0.93 (d, ³J_{HH}=9.3 Hz, 2H); 0.68 (s, 1H); 0.63 (s, 9H).

¹³C-NMR (75 MHz, CDCl₃): δ=210.9; 170.2; 165.2; 151.7; 147.6; 137.1; 125.1; 125.0; 123.3; 122.8; 120.1; 118.3; 115.3; 113.1; 112.9; 108.9; 98.5; 58.9; 57.3; 57.1; 47.3; 42.4; 40.4; 34.5; 32.3; 31.1; 27.4; 24.2; 23.5.

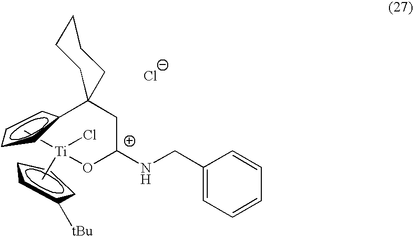

M.p.: 143° C.

¹H-NMR (400 MHz, CDCl₃): δ=12.58 (s, 1H), 7.42 (d, ³J=7.1 Hz, 2H), 7.31 (t, ³J=7.1 Hz, 2H), 7.25 (t, ³J=7.1 Hz, 1H), 6.94 (brs, 1H), 6.86-6.64 (m, 2H), 6.52 (s, 1H), 6.27 (s, 1H), 6.13 (s, 1H), 5.87 (brs, 1H), 4.45 (d, ²J=12.7 Hz, 1H), 4.29 (d, ²J=13.0 Hz, 1H), 3.43 (brs, 1H), 2.48 (brs, 1H), 1.97-1.88 (brs, 1H), 1.88 (brs, 1H), 1.66 (brs, 2H), 1.59-1.39 (m, 3H), 1.36-1.22 (m, 3H), 1.18 (s, 9H), 1.11-0.96 (m, 1H). ¹³C-NMR (100 MHz, CDCl₃): δ=176.7, 150.8, 149.6, 136.9, 128.7, 128.5, 128.0, 127.4, 124.1, 123.6, 123.6, 115.2, 114.7, 113.4, 111.5, 48.1, 45.2, 38.8, 38.6, 34.3, 34.0, 30.9, 25.3, 22.4, 21.8.

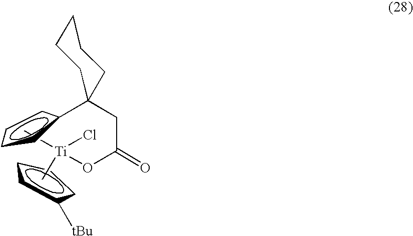

M.p.: 212° C.

¹H-NMR (400 MHz, CDCl₃): δ=6.82 (dd, ³J=4.6 Hz, ⁴J=2.5 Hz, 1H), 6.59 (dd, ³J=8.3 Hz, ⁴J=2.3 Hz, 2H), 6.54 (dd, ³J=4.5 Hz, ⁴J=2.3 Hz, 1H), 6.26 (dd, ³J=6.2 Hz, ⁴J=2.6 Hz, 2H), 5.98-5.90 (m, 2H), 2.72 (d, ²J=13.0 Hz, 1H), 2.37 (d, ²J=13.0 Hz, 1H), 2.00-1.30 (m, 10H), 1.27 (s, 9H).

¹³C-NMR (100 MHz, CDCl₃): δ=175.2, 151.8, 145.7, 126.4, 121.7, 116.0, 115.5, 115.2, 114.9, 114.4, 107.9, 47.8, 37.9, 37.7, 36.9, 34.0, 30.6, 25.8, 22.0.

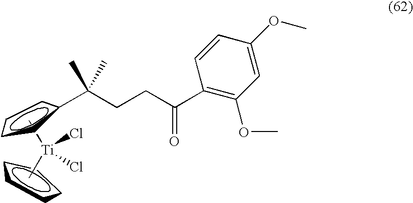

M.p.: 120° C. (decomposition)

¹H-NMR (400 MHz, CDCl₃): δ=7.70 (d, ³J=8.7 Hz, 1H), 6.62 (dd, ³J=2.6 Hz, ³J=2.6 Hz, 2H), 6.55 (s, 5H), 6.52 (s, 1H), 6.49 (dd, ³J=2.6 Hz, ³J=2.6 Hz, 2H), 6.42 (d, ³J=2.0 Hz, 1H), 3.83 (s, 6H), 2.66 (dd, ³J=8.1 Hz, ³J=8.1 Hz, 2H), 1.80 (dd, ³J=8.2 Hz, ³J=8.3 Hz, 2H), 1.36 (s, 6H). ¹³C-NMR (100 MHz, CDCl₃): δ=200.4, 164.5; 160.7, 148.4, 132.7, 121.1, 120.4, 119.2, 119.1, 105.3, 98.4, 55.7, 55.6, 41.2, 39.1, 37.1, 26.8.

### REFERENCES

1. Y. A. Hannun (1997), Apoptosis and the dilemma of cancer therapy. Blood 89,
2. J. A. Hickmann (1996), Apoptosis and chemotherapy resistance, Eur. J. Cancer 32A, 921-926
3. M. Raisova, M. Bektas, T. Wieder, P. T. Daniel, J. Eberle, C. E. Orfanos, C. C. Geilen (2000). Resistance to CD95/Fas-induced and ceramide-mediated apoptosis of human melanoms cells is caused by a defective mitochondrial cytochrom c release, FEBS Lett. 473. 27-32
4. G. M. Cohen (1997), Caspases:the executioners of apoptosis, Bjochem. J. 326, 1-16
5. F. Eβmann, T. Wieder, A. Otto, E.-C. Müller, B. Dörken, P. T. Daniel (2000). The GDP dissociation inhibitor, D4-GDI (Rho-GD I 2), but not the homologous Rho-GDI1, is cleaved by caspase-3 during drug-induced apoptosis, Biochem. J.
6. T. Wieder, C. Perlitz, M. Wieprecht, R. T. C. Huang, C. C. Geilen, C. E. Orfanos (1995), Two new sphingomyelin analogues inhibit phosphatidylcholine biosynthesis by decreasing membranebound CTP: phosphocholine cytydyltransferase levels in HaCaT cells, Biochem. J. 331, 873-879
7. T. Wieder, F. Eβmann, A. Prokop, K. Schmelz, K. Schulze-Osthoff. R. Beyaert, B. Dörken, P. T. Daniel (2001), Activation of caspase-8 in drug-induced apoptosis of B-lymphoid cells is independent of CD95/Fas receptor-ligand interaction and occurs downstream of caspase-3, Blood 97, 1378-1387.
8. T. Wieder, A. Prokop, 8. Bagci, F. Essmann, D. Bernicke, K. $chulze-Osthoff, B. Dörken, H. G. Schmalz, P. T. Daniel, G. Henze, Piceatannol, ahydroxylated analog of the chemopreventive agent Resveratrol, is a potent inducer of apoptosis in the lymphoma cell line BLAB and in primary, leukemic lymphoblasts. Leukemia 2001, 15:1735-1742
9. A. Prokop, T. Wieder, I. Sturm, F. Eβmann. K. Seeger, C. Wuchter. W.-D. Ludmwig, G. Henze, B. Dörken, P. T. Daniel (1999), Relapse in childhood acute lymphoblastic leukemia is associated with decrease of Bax/BCL-2-ratio and 108s of spontaneous caspase-3 processing in vivo, Leukemia (2000), 14:1606-1613
10. Prokop, A. Wrasidlo, W., Lode, L., Herold, R., Lang, F., Henze, G., Dörken, B. Wieder, T. end Daniel, P. T., Induction of apoptosis by enediyne antibiotic calicheamicin JII proceeds through a caspase-mediated mitochondrial amplification loop in an entirely Bax-dependent manner. Oncogene (2003), 22 (57): 9107-20.
11. Schlawe, O., Majdalani. A., Velcicky, J., Heβler, E., Wieder, T., Prokop, A., and Schmalz, H-G., (2004) Iron-containing nucleoside analogs with pronounced apoptosis-inducing activity. Angewandte Chemie, 116, 1763-1766
12. Diller, R. A., Riepl, H. M., Rose, O. Fries, C., Henze, G. and Prokop, A., Synthesis of demethylxanthohumol, a new potent apoptosis inducing agent from hops, Chemistry & Biodiversity, (2005), 2, 1331-1338
13. Gansäuer, A.; Franke, D.; Lauterbach, T., Nieger, M. (2005) A modular and efficient synthesis of functional titanocenes J. Am. Chem. Soc. 127, 11622-11623.

1. A medicament comprising at least one compound of formula (Ia) and formula (Ib): 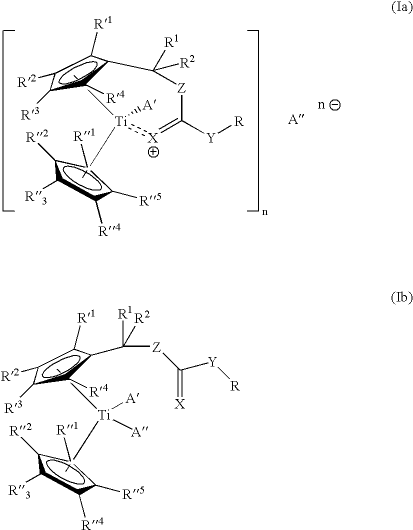
wherein
Z is selected from the group consisting of a covalent bond; at least one at least divalent hetero atom; a saturated, unsaturated, branched, unbranched and/or cyclic substituted or unsubstituted hydrocarbon chain; a saturated or unsaturated, branched, unbranched and/or cyclic substituted or unsubstituted hydrocarbon chain provided with hetero atoms in the chain thereof, and combinations thereof;
R′¹ to R′⁴ and R″¹ to R″⁵ independently are H; at least one hetero atom; a saturated, unsaturated, branched, unbranched and/or cyclic; substituted or unsubstituted hydrocarbon chain; a saturated or unsaturated, branched, unbranched and/or cyclic, substituted or unsubstituted hydrocarbon chain provided with hetero atoms in the chain thereof, or combinations thereof;
wherein the two cyclopentadienyl rings of formulas Ia and Ib are optionally linked together via any of residues R″¹ to R″⁵;
R¹ and R² independently are H; a hetero atom; a saturated, unsaturated, branched, unbranched and/or cyclic, substituted or unsubstituted hydrocarbon chain; a saturated or unsaturated, branched, unbranched and/or cyclic substituted or unsubstituted hydrocarbon chain provided with hetero atoms in the chain thereof; or combinations thereof;
A′ and A″ are independently F, Cl, Br, I and/or a physiologically acceptable acid residue of an organic or inorganic acid;
n is a positive integer;
X is O, S, NH, NR³ or NH⁴R⁵, wherein R³, R⁴ and R⁵ independently have the same meanings as R′¹ to R′⁴;
Y is a covalent bond, O, S or NR⁶, wherein R⁶ has the same meaning as R′¹ to R′⁴;
R is a saturated, unsaturated, branched, unbranched and/or cyclic substituted or unsubstituted hydrocarbon chain provided that one or more carbons in the hydrocarbon chain may be optionally substituted with a hetero atom.

2. The medicament according to claim 1, wherein X is O.

3. The medicament according to claim 1, wherein Y is NR⁶.

4. The medicament according to, claim 1 wherein X is O and Y is NR⁶.

5. The medicament according to, claim 1 wherein Z is CR³R⁴, and wherein R³ and R⁴ independently have the meanings as mentioned in claim 1 for R′¹ to R′⁴.

6. The medicament according to, claim 1 wherein A′ and A″ are independently chloride or bromide.

7. The medicament according to, claim 1 wherein R′¹ to R′⁴ is a hydrogen atom.

8. The medicament according to claim 1, wherein R″¹ to R″⁵ is a hydrogen atom.

9. The medicament according to claim 1, wherein the medicament comprises a compound of formula (II): 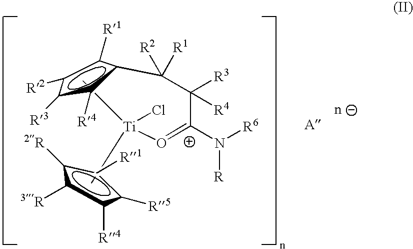
.

10. The medicament according to claim 9, wherein the medicament comprises a compound of formula (III): 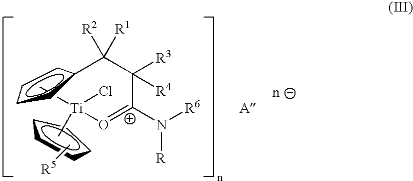
.

11. The medicament according to claim 9, wherein the medicament comprises a compound of formula (IVa) or (IVb): 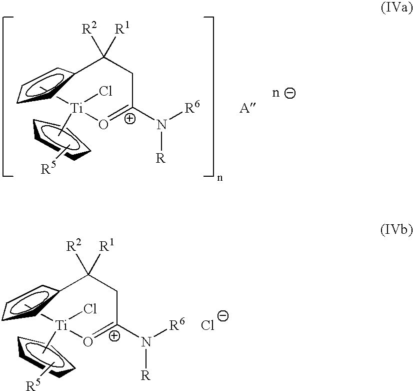
.

12. The medicament according to claim 9 wherein the medicament comprises a compound of formula (Va), (Vb), (Vc) or (Vd): 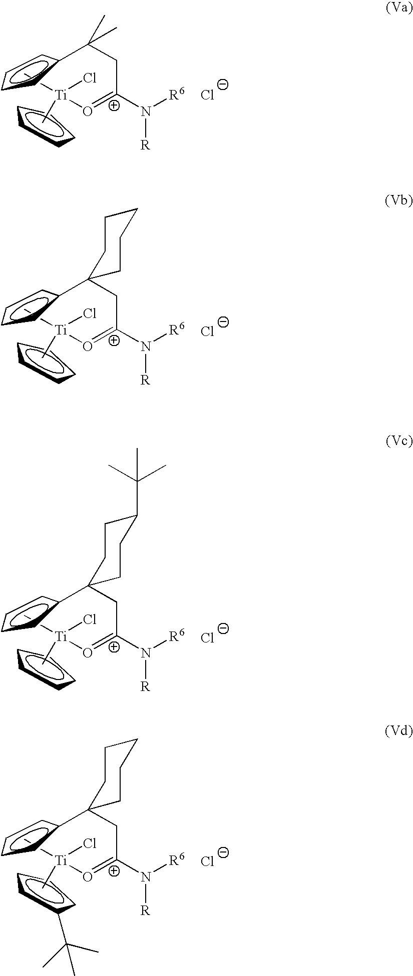
.

13. The medicament according to claim 9, wherein the medicament comprises a compound of formula (VI): 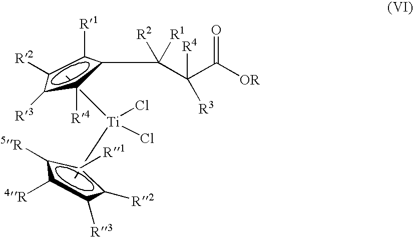
.

14. The medicament according to claim 13, wherein the medicament comprises a compound of formula (VIIa) or (VIIb): 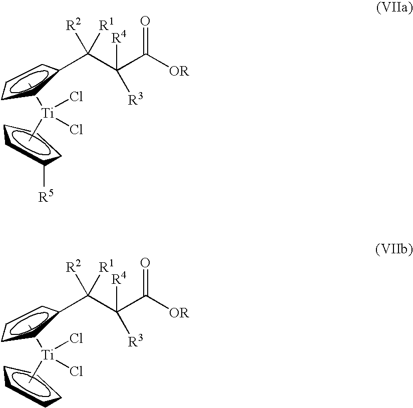
.

15. The medicament according to claim 13, wherein the medicament comprises a compound of formula (VIIIa) or (VIIIb): 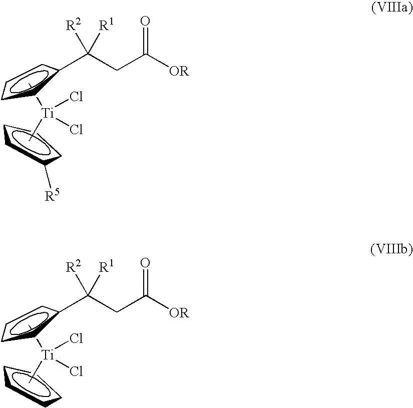
.

16. The medicament according to claim 9, wherein the medicament comprises a compound of formulas (IXa) or (IXb): 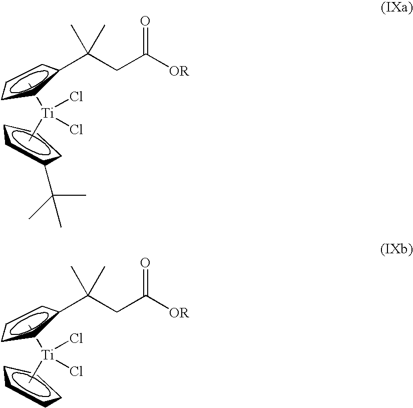
.

17. The medicament according to claim 1, wherein the medicament comprises a compound of formula (X): 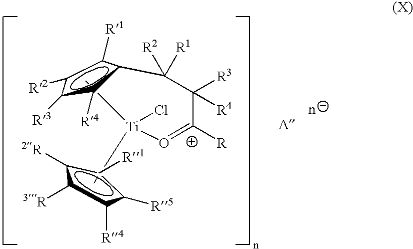
.

18. The medicament according to claim 17, wherein the medicament comprises a compound of formula (XI): 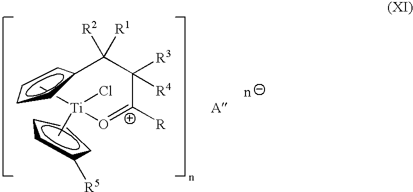
.

19. The medicament according to claim 17, wherein the medicament comprises a compound of formula (XII): 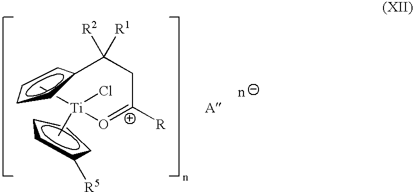
.

20. The medicament according to claim 17, wherein the medicament comprises a compound of formula (XIIIa), (XIIIb), (XIIIc) or (XIIId):

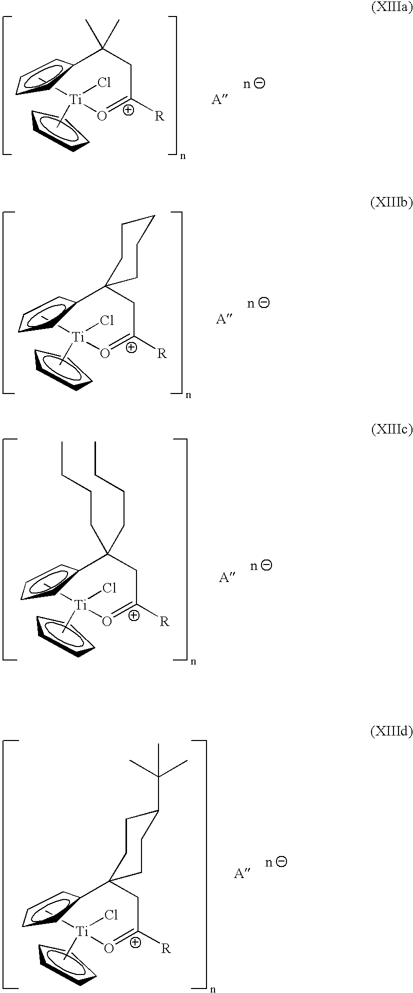

21. The medicament according to claim 1, wherein the medicament comprises at least one of the substances shown below: 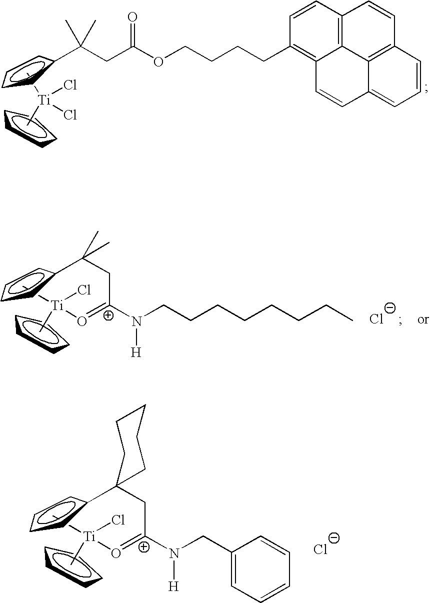
.

22. A compound having the structural formula (Ia) or (Ib) of claim 1, except for the compounds having the following structures:

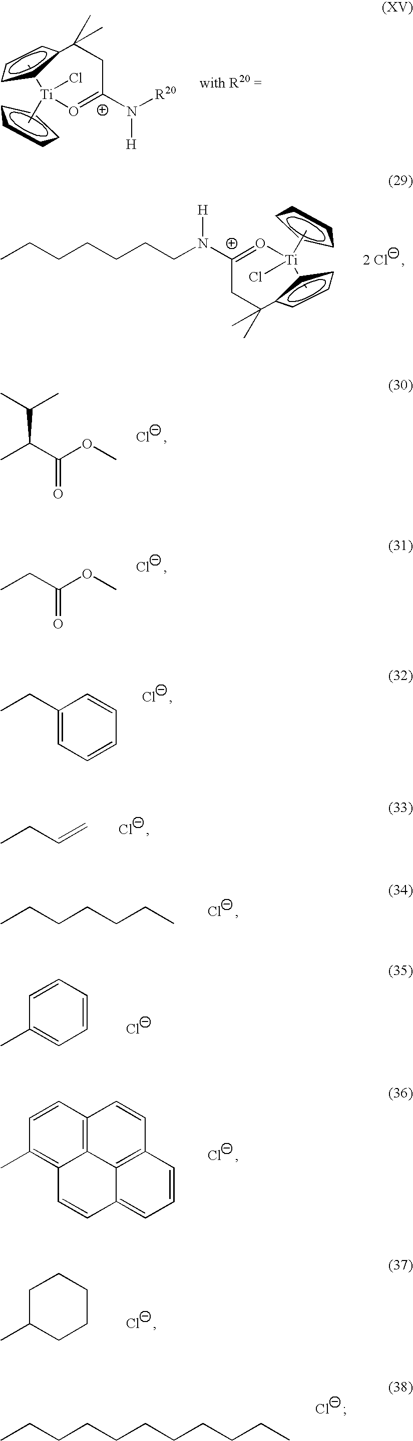

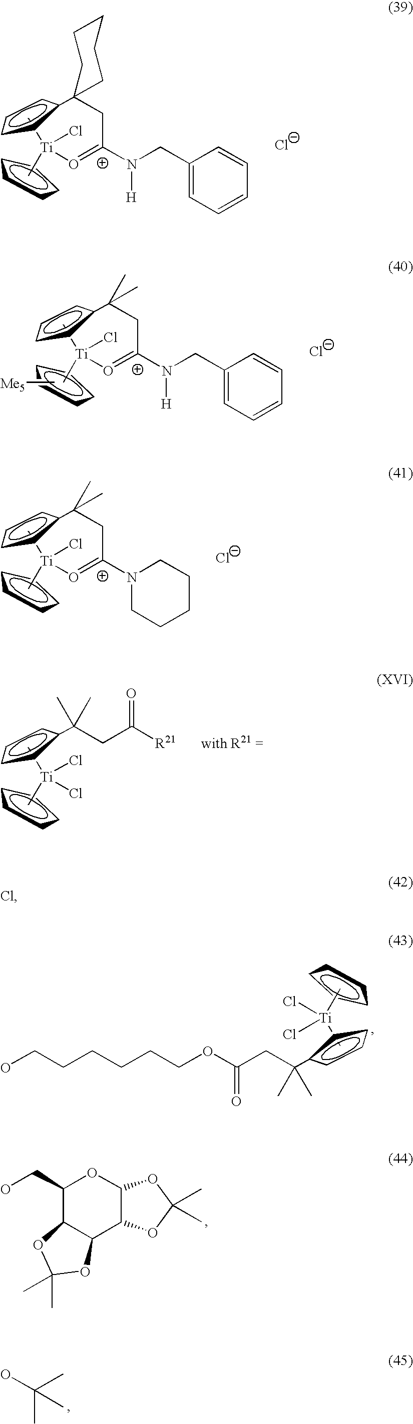

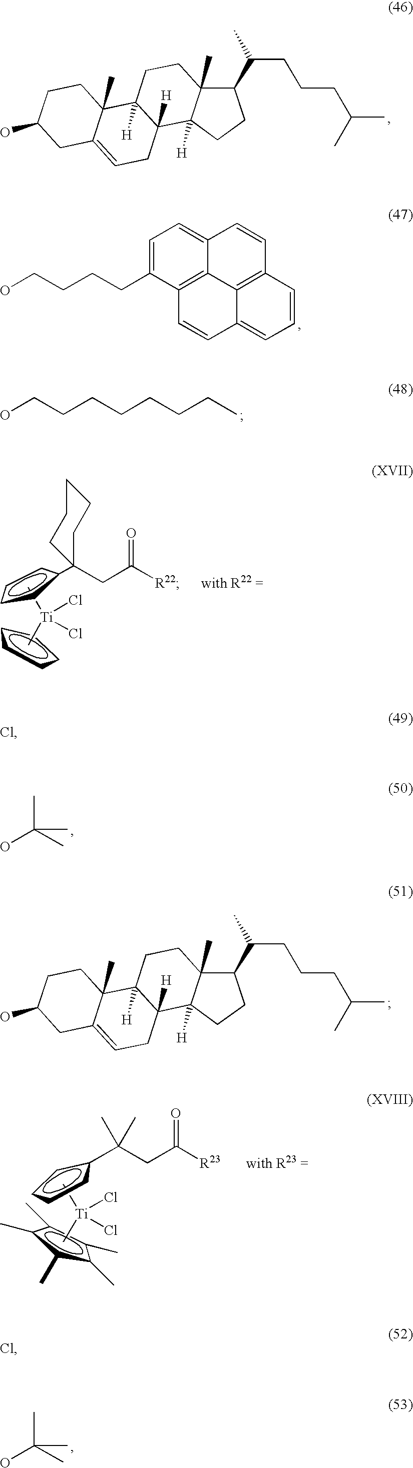

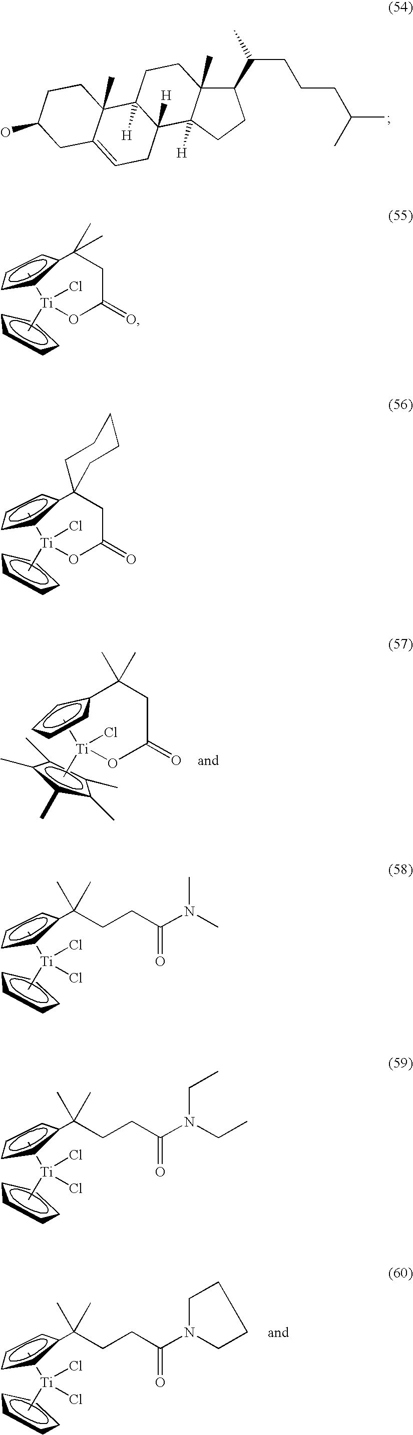

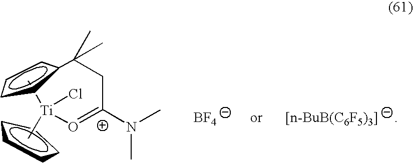

23. The compound according to claim 22 having the structural formula (II): 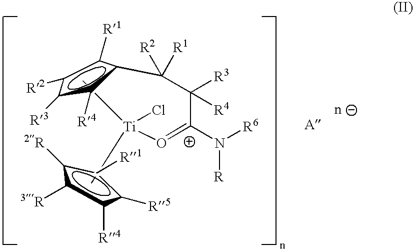
.

24. The compound according to claim 22 having the structural formula (VI): 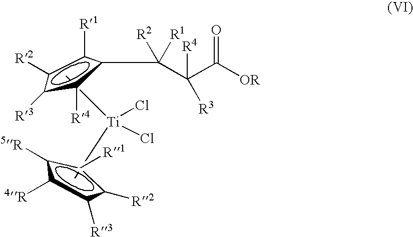
.

25. The compound according to claim 22 having the structural formula (III): 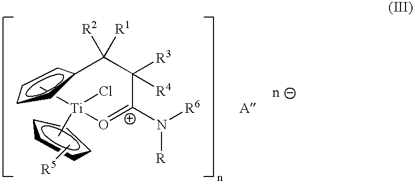
.

26. The compound according to claim 22 having the structural formulas (IVa) or (IVb): 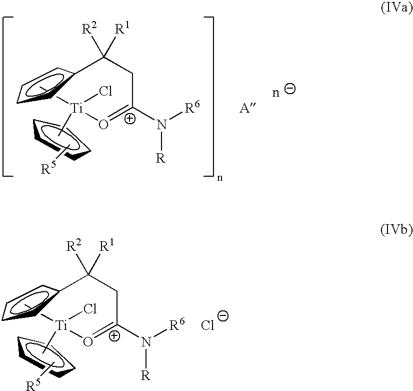
.

27. The compound according to claim 22 having the structural formulas (Va), (Vb), (Vc) or (Vd): 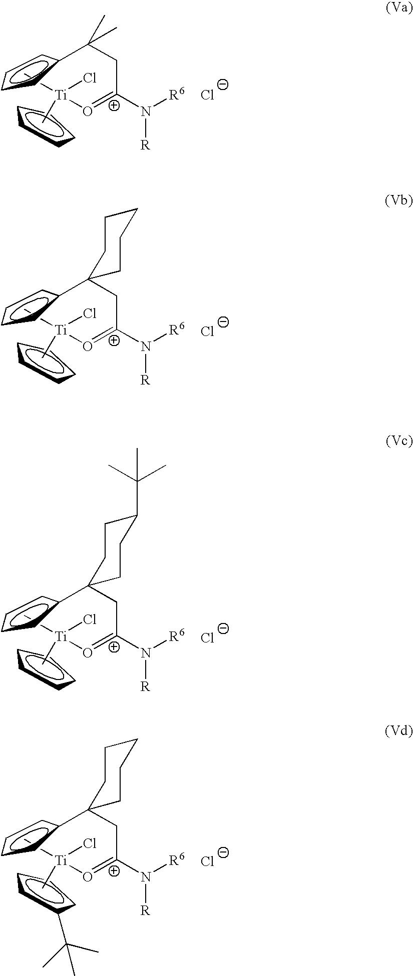
.

28. The compound according to claim 24 having the structural formulas (VIIa) or (VIIb): 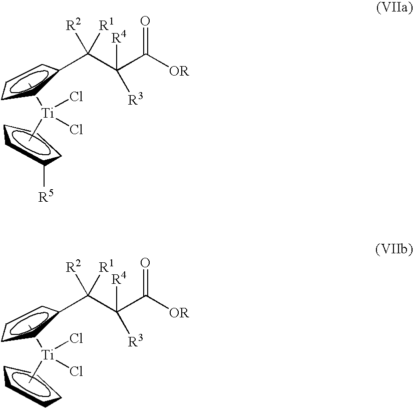
.

29. The compound according to claim 24 having the structural formulas (VIIIa) or (VIIIb): 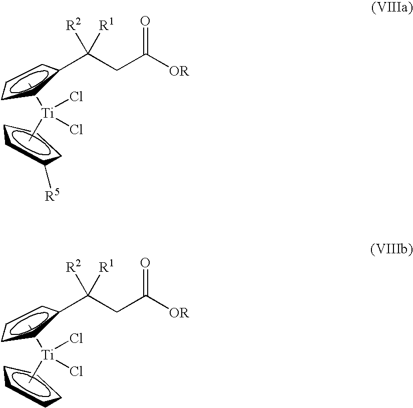
.

30. The compound according to claim 24 having the structural formulas (IXa) or (IXb): 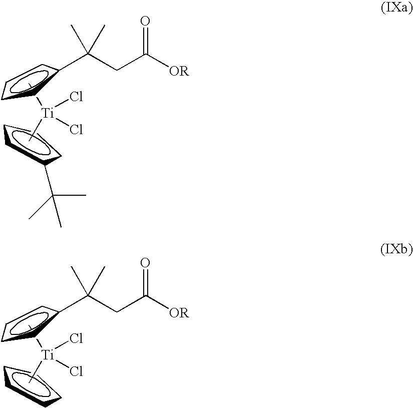
.

31. The compound according to claim 22 having the structural formula (X), wherein Y is a covalent bond: 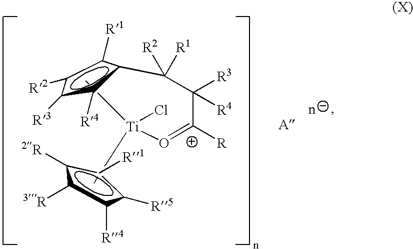
.

32. The compound according to claim 31 having the structural formula (XI): 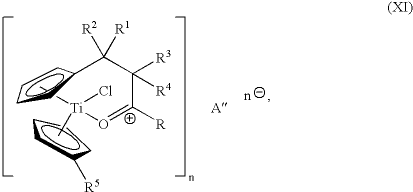
.

33. The compound according to claim 31 having the structural formula (XII): 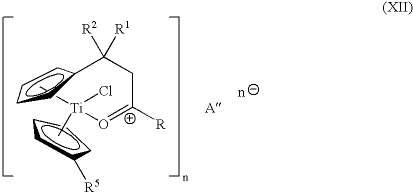
.

34. The compound according to claim 31 having the structural formulas (XIIIa), (XIIIb), (XIIIc) or (XIIId) 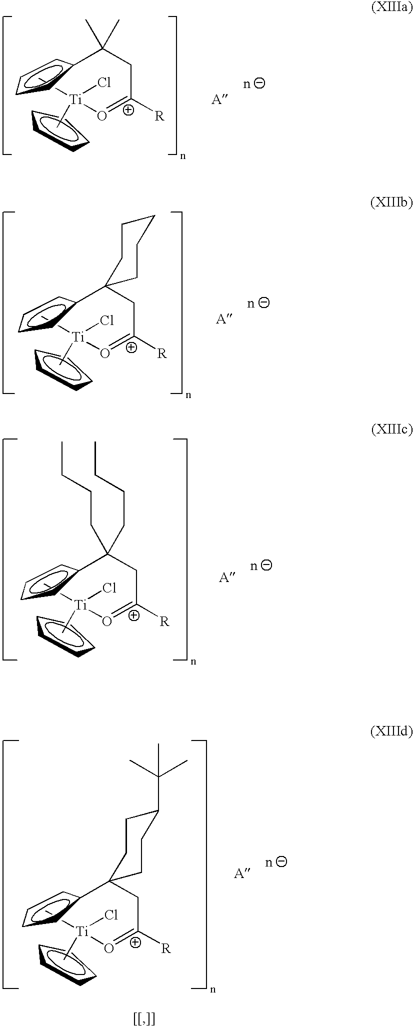
.

35. A method of treating a disease related to fast-proliferating cells comprising administering the medicament of claim 1 to a patient in need thereof.

36. The method according to claim 35, wherein said disease related to fast-proliferating cells is selected from the group consisting of malignant diseases of the bone marrow, other hematopoietic organs, solid tumors, sarcomas, epithelial tumors, benign and semimalignant fast-proliferating tumors, skin diseases, keloids, basaliomas, lymphomas, inflammatory, chronic inflammatory, bacterial and auto-immune diseases.

37. The method according to claim 35 for antibacterial, antimycotic, antiprotozoan, antiplasmodium, antiviral, antihelminthic or immunosuppressant therapies.

38. The method according to claim 35 for the treatment of tumor diseases and leukemias, for the therapy of tumors of different origin, such as epithelial tumors, malignant diseases of the skin and for the therapy of malignant brain tumors, such as medulloblastoma, astrocytoma and/or glioblastoma.

39. A process for the preparation of a compound according to claim 22, comprising:
(a) reacting a cyclopentadienyl derivative with a carbonyl compound to form a fulvene,
(b) adding an ester enolate to the product of step (a): 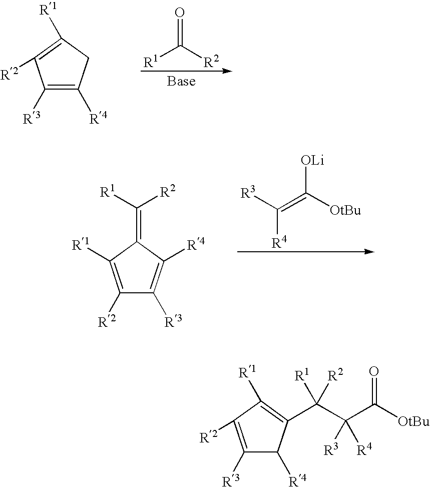
(c) deprotonation of the product of step (b) with a string base;
(d) metallization with cyclopentadienyltitanium trichloride; and
(e) heating or treating a product of step (d) with ZnCl₂ to form cyclic carboxylate C according to the reaction scheme shown below:

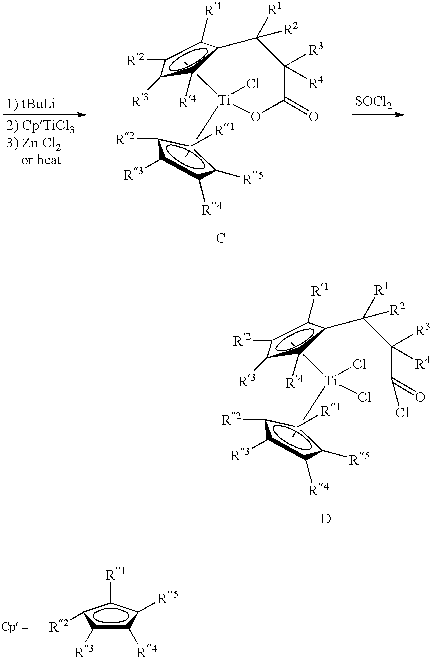

40. A diagnostic agent for the diagnosis of fast-proliferating cells, comprising a compound of formula (Ia) and/or (Ib) of claim 1.

41. A composition comprising the compound according to formula (Ia) and/or (Ib) of claim 1 and a cytostatic agent.

42. The composition according to claim 41, wherein said cytostatic agent is selected from the group consisting of daunorubicin, doxorubicin, epirubicin, idarubicin, vincristin and cytarabin.

43. The composition according to claim 42, wherein said cytostatic agent is a nucleoside analogue.

44. The medicament of claim 1, wherein said hetero atom is oxygen, nitrogen or halogen.

45. The process of claim 39, wherein said ester enolate is the enolate of tert-butyl acetate.

46. The composition of claim 43, wherein said nucleoside analogue is cytarabin (AraC).
